(19) United States
(12) Patent Application Publication
(10) Pub. No.: US 2026/0053895 A1
(43) Pub. Date: Feb. 26, 2026

(54) PHARMACEUTICAL COMPOSITIONS AND THEIR USES, AND METHODS FOR IMPROVING THE STORAGE STABILITY OF PHARMACEUTICAL COMPOSITIONS

(71) Applicants: Chuanhui XIE, Shanghai (CN); Rongying CHEN, Shanghai (CN); Hongliang SUN, Shanghai (CN)

(72) Inventors: Chuanhui XIE, Shanghai (CN); Rongying CHEN, Shanghai (CN); Hongliang SUN, Shanghai (CN)

(21) Appl. No.: 19/384,871

(22) Filed: Nov. 10, 2025

Related U.S. Application Data
(63) Continuation of application No. PCT/CN24/92456, filed on May 10, 2024.

(30) Foreign Application Priority Data
May 10, 2023 (CN) 202310524629.5

(51) Int. Cl. A61K 38/26; A61K 9/00; A61K 38/28; A61K 47/02; A61K 47/10; A61K 47/26

(52) U.S. Cl. CPC A61K 38/26; A61K 9/0019; A61K 38/28; A61K 47/02; A61K 47/10; A61K 47/26

(57) ABSTRACT

The application provides embodiments of stable pharmaceutical compositions, a method for improving the storage stability of pharmaceutical compositions, and uses of the stable pharmaceutical compositions. The pharmaceutical composition comprises: a first active ingredient comprising a GLP-1 receptor agonist, and a surfactant. In certain embodiments, the surfactant comprising an alkyl glycoside. In certain embodiments, one or more alkyl glycosides (e.g., without limitation, DDM) inhibit the formation of GLP-1 fibrosis in pharmaceutical compositions comprising GLP-1 receptor agonists such as GLP-1 or its analogues or derivatives of GLP-1 analogues, and thereby improving physical stability.

### PRIORITY CLAIM

This application claims priority to Chinese patent Application No. 2023105246295, filed May 10, 2023, which claims priority herein in its entirety.

### REFERENCE TO ELECTRONIC SEQUENCE LISTING

The application contains a Sequence Listing which has been submitted electronically in .XML format and is hereby incorporated by reference in its entirety. Said .XML copy, created on Jul. 18, 2024, is named “057783.8023.WO00.xml” and is 23,445 bytes in size. The sequence listing contained in this .XML file is part of the specification and is hereby incorporated by reference herein in its entirety.

### TECHNICAL FIELD

The present disclosure relates to the field of biotech/pharmaceuticals, and more specifically, the application relates to a pharmaceutical composition comprising a GLP-1 receptor agonist and a method for improving the storage stability of the pharmaceutical composition and the use of the pharmaceutical composition in the preparation of a medicament.

### BACKGROUND

About 120 million people worldwide have diabetes. Exogenous insulin is mainly used to treat diabetes. Insulin is the only hormone in the body that lowers blood sugar and promotes glycogen, fat, and protein synthesis. Insulin glargine is an analogue of human insulin and can be obtained by recombinant DNA technology. After insulin glargine is injected into the subcutaneous tissue, the fine deposits formed by the neutralization of the acidic solution continue to release a small amount of insulin glargine, resulting in a predictable, long-acting, smooth, and peakless plasma concentration/time characteristic.

Glucagon-like peptide 1 (hereinafter referred to as GLP-1) is an incretin secretion peptide, which has the following physiological effects: glucose concentration-dependent insulin secretion, thereby reducing the risk of hypoglycemia; promoting insulin synthesis in vivo, promoting the differentiation and generation of p cells, inhibiting the release of glucagon, inhibiting gastric emptying and feeding impulse, and improving the sensitivity to insulin receptors. It has been clinically proven to have advantages such as hypoglycemia, weight loss, and increased cardiovascular benefits.

The combination of insulin and GLP-1 receptor agonists can take full advantage of the complementary clinical advantages of the two, such as glucose reduction, weight loss, and reduction of hypoglycemia risk and insulin resistance. Premixed compositions comprising basal insulin and GLP-1 are easier to administer, and therefore have a significant market demand.

However, the current pharmaceutical compositions of GLP-1 receptor agonists still have room for improvements.

### SUMMARY

One aspect of the invention relates to a stable pharmaceutical composition comprising a first active ingredient comprising one or more GLP-1 receptor agonists; and one or more surfactants (e.g., without limitation, alkyl glycosides (e.g., without limitation, n-dodecyl β-D-maltoside (DDM))). Embodiments of the stable pharmaceutical compositions disclosed herein showed improved stability of the one or more GLP-1 receptor agonists. In certain embodiments, the improved stability relates to physical stabilities. In certain embodiments, the stable pharmaceutical compositions are stable liquid pharmaceutical compositions. In certain embodiments, the stable liquid pharmaceutical compositions are stable aqueous pharmaceutical compositions. In certain embodiments, the stable aqueous pharmaceutical compositions are stable aqueous injectable pharmaceutical compositions. In certain embodiments, the stable aqueous injectable pharmaceutical compositions are single-dose or multi-dose aqueous injectable pharmaceutical compositions. Examples of the one or more GLP-1 receptor agonists include, without limitation, GLP-1, GLP-1 analogs, and derivatives of GLP-1 analogs.

In certain embodiments, the stable pharmaceutical composition further comprises a second active ingredient comprising insulin, insulin analogs, and/or derivatives of insulin analogs, and may be referred to as a stable combination pharmaceutical composition herein. Embodiments of the stable combination pharmaceutical composition disclosed herein also have high stability of the first and the second active ingredients. In certain embodiments, the stability relates to physical stabilities. In certain embodiments, the stable combination pharmaceutical compositions are stable liquid combination pharmaceutical compositions. In certain embodiments, the stable liquid combination pharmaceutical compositions are stable aqueous combination pharmaceutical compositions. In certain embodiments, the stable aqueous combination pharmaceutical compositions are stable aqueous injectable combination pharmaceutical compositions. In certain embodiments, the stable aqueous injectable combination pharmaceutical compositions are single-dose or multi-dose aqueous injectable combination pharmaceutical compositions.

Another aspect of the invention relates to a method for improving the storage stability of a pharmaceutical composition comprising a first active ingredient comprising one or more GLP-1 receptor agonists. In certain embodiments of the method disclosed herein, the method comprises mixing one or more surfactants (e.g., without limitation, alkyl glycosides (e.g., without limitation, DDM)) with the pharmaceutical composition. In certain embodiments, the pharmaceutical composition is a combination pharmaceutical composition further comprising a second active ingredient comprising insulin, insulin analogs, and/or derivatives of insulin analogs.

Another aspect of the invention relates to the use of the stable pharmaceutical composition for the manufacture of a medicament for use in: (i) preventing and/or treating diabetes, and/or for reducing HbA1C; (ii) delaying or preventing the progression of diabetes, delaying the progression of impaired glucose tolerance to insulin-requiring type 2 diabetes, delaying or preventing insulin resistance, and/or delaying the progression of type 2 diabetes that does not require insulin to insulin-requiring type 2 diabetes; (iii) improving β-cell function, and/or restoring β-cell glucose sensitivity; (iv) preventing and/or treating eating disorders; reducing gastric motility; delaying gastric emptying; (v) preventing and/or treating complications of obesity disease; (vi) preventing and/or treating complications of diabetes; and/or (vii) preventing and/or treating cardiovascular disease. In certain embodiments, the stable pharmaceutical composition is a stable combination pharmaceutical composition further comprising a second active ingredient comprising insulin, insulin analogs, and/or derivatives of insulin analogs.

Another aspect of the invention relates to the stable pharmaceutical composition for use in: (i) the prevention and/or treatment of diabetes, and/or for the reduction of HbA1C; (ii) the delay or prevention of the progression of diabetes, the delay of the progression of impaired glucose tolerance to insulin-requiring type 2 diabetes, the delay or prevention of insulin resistance, and/or the delay of the progression of type 2 diabetes that does not require insulin to insulin-requiring type 2 diabetes; (iii) the improvement of 3-cell function, and/or the restoration of j-cell glucose sensitivity; (iv) the prevention and/or treatment of eating disorders; the reduction of gastric motility; the delay of gastric emptying; (v) the prevention and/or treatment of complications of obesity disease; (vi) the prevention and/or treatment of complications of diabetes; and/or (vii) the prevention and/or treatment of cardiovascular disease. In certain embodiments, the stable pharmaceutical composition is a stable combination pharmaceutical composition further comprising a second active ingredient comprising insulin, insulin analogs, and/or derivatives of insulin analogs.

Another aspect of the invention relates to methods for (i) preventing and/or treating diabetes, and/or for reducing HbA1C; (ii) delaying or preventing the progression of diabetes, delaying the progression of impaired glucose tolerance to insulin-requiring type 2 diabetes, delaying or preventing insulin resistance, and/or delaying the progression of type 2 diabetes that does not require insulin to insulin-requiring type 2 diabetes; (iii) improving β-cell function, and/or restoring β-cell glucose sensitivity; (iv) preventing and/or treating eating disorders; reducing gastric motility; delaying gastric emptying; (v) preventing and/or treating complications of obesity disease; (vi) preventing and/or treating complications of diabetes; and/or (vii) preventing and/or treating cardiovascular disease in a subject comprising administering to the subject a therapeutically effective amount of the stable pharmaceutical composition. In certain embodiments, the stable pharmaceutical composition is a stable combination pharmaceutical composition further comprising a second active ingredient comprising insulin, insulin analogs, and/or derivatives of insulin analogs.

### BRIEF DESCRIPTION OF THE DRAWINGS

The above and/or additional aspects and advantages of the present disclosure will become apparent and readily understood from the description of the embodiments taken in conjunction with the following drawings, in which:

FIG. 1 shows the ThT test results of the insulin glargine/GLP-1 combination composition according to one embodiment of the present disclosure;

FIG. 2 shows the ThT test results of beinaglutide stock solution according to one embodiment of the present disclosure;

FIG. 3 shows the ThT test results of beinaglutide compositions without and with DDM at different concentrations according to one embodiment of the present disclosure;

FIG. 4 shows the ThT test results of insulin glargine/GLP-1-DDM combination composition comprising different bacteriostatic agents according to one embodiment of the present disclosure;

FIG. 5 shows the ThT test results of the insulin glargine/GLP-1 combination composition according to one embodiment of the present disclosure; and

FIG. 6 (SEQ ID NO: 5) shows physical stabilities of GLP-1 pharmaceutical compositions with 0.1% DDM and without DDM according to several embodiments of the present disclosure.

FIG. 7 shows physical stabilities of P13 (peptide sequence: ELAEGTFTSDLSKQMEEEAVRLFIEWLKNGGPSSGAPPPS-NH₂ (SEQ ID NO: 3), a GLP-1/GIP dual agonist) pharmaceutical compositions with 0.1% DDM and without DDM according to several embodiments of the present disclosure.

FIG. 8 shows of physical stabilities of 988927 (peptide sequence: HGEGTFTSDSSSYLEEQAAKEFIAWLVKGRG (SEQ ID NO:4)) with 0.1% DDM and without DDM according to several embodiments of the present disclosure.

FIG. 9 shows GLP-1 solubility over phenol concentration in the presence of DDM or without.

FIG. 10 shows plasma concentration of insulin aspart in beagle after administration of an insulin aspart only composition and an insulin aspart/GLP-1 combination composition according to an embodiment of the present disclosure.

FIG. 11 shows plasma concentration of GLP-1 in beagle after administration of a GLP-1 only composition and an insulin aspart/GLP-1 combination composition according to an embodiment of the present disclosure.

### DETAILED DESCRIPTION

The embodiments of the present disclosure are described in detail below. The embodiments described below with reference to the drawings are exemplary and are intended to explain the present disclosure, but cannot be understood as limiting the present disclosure.

### Definition

Unless otherwise specified, the term “active ingredient” used herein refers to a substance or combination of substances in a drug that produces a therapeutic or preventive effect. For example, according to the embodiments of the present application, the active ingredient used may be one or Various GLP-1 receptor agonists can also be additionally added with insulin or its analogs or derivatives in pharmaceutical compositions, so that the pharmaceutical composition can exert the desired therapeutic or preventive effect when applied to the subject.

Unless otherwise specified, the term “surfactant” used in this article refers to a class of chemical substances that can reduce the surface tension and interfacial tension of liquids and are usually composed of hydrophobic groups and hydrophilic groups. Surfactants are usually used to adjust the solubility, stability and bioavailability of drugs, thereby affecting the absorption, distribution, metabolism and excretion of drugs.

Unless otherwise specified, the term “alkyl glycosides” used in this article is a type of surfactant composed of sugar groups and alkyl chains. It has good surface activity and biodegradability. These compounds usually It is obtained by enzymatically catalyzing the reaction of sugars and fatty alcohols at a certain temperature and pressure. Since the molecular structure of alkyl glycosides comprises both hydrophobic and hydrophilic groups, they have properties similar to traditional surfactants. Among them, the alkyl chain is hydrophobic and can interact with non-polar solvents; while the sugar group is hydrophilic and can interact with water. The inventor of the present disclosure found that when preparing the pharmaceutical composition of the present application, alkyl glycosides (e.g., without limitation, DDM) can be used as non-ionic surfactants to promote the stability, solubility and bioavailability of the drug, and can also Reduce the toxicity and irritation of preparations and improve the safety and tolerability of drugs.

Unless otherwise specified, the term “analogue” used herein refers to a molecule that is similar in structure to the original molecule but has the same or different chemical properties. For example, in drug research, researchers might design and synthesize a series of compounds that are structurally similar to a known drug molecule but have different chemical groups or substituents that alter its pharmacological properties. Such as absorption, metabolism, drug efficacy, etc. These analogs may have better pharmacological properties or toxicological properties and therefore have the potential for further development as drugs. More specifically, in this application, “analog” is used to mean a peptide in which one or more amino acid residues of the parent peptide have been replaced by other amino acid residues and/or in which one or more amino acid residues of the parent peptide Residues have been deleted and/or one or more amino acid residues have been added to the parent peptide. Such additions typically occur at the N-terminus or C-terminus or both of the parent peptide. According to specific embodiments, generally “analogs” in this application are peptides in which 6 or less amino acids of the parent peptide have been substituted and/or added and/or deleted, more preferably such peptides, wherein 3 or less amino acids of the parent peptide have been substituted and/or added and/or deleted, most preferred are peptides in which one amino acid of the parent peptide has been substituted and/or added and/or deleted.

Unless otherwise specified, the term “derivative” used in this article refers to a new molecule obtained from an original molecule through chemical modification or transformation. For example, in drug research, researchers may start from a known drug molecule and synthesize a series of new compounds by changing, substituting, or modifying its structure. These new molecules are called compounds of the drug molecule. derivative. Derivatives may have better pharmacological properties, better safety or lower side effects and therefore also play an important role in drug development. Specifically, in this application, “derivative” is used to represent a peptide in which one or more amino acid residues of the parent peptide have been introduced into substituents. Typical substituents can be amides, sugars, alkyl, Acyl, ester, PEGylation, etc.

Unless otherwise specified, the term “GLP-1 receptor agonists” used in this article refers to a class of drugs that treat diabetes by mimicking glucagon-like polypeptide-1 (GLP-1) biological activity to promote the secretion of insulin and inhibit the production and release of glucose, thereby helping to lower blood sugar levels. Among them, GLP-1 is a peptide hormone secreted by intestinal L cells and brainstem neurons. It has a variety of biological functions, including suppressing appetite, stimulating insulin secretion, inhibiting glucose production and release, etc. GLP-1 receptor agonists can stimulate GLP-1 receptors, enhance insulin secretion, and inhibit glucose production and release. Due to their effectiveness in glycemic control and good tolerability, GLP-1 receptor agonists have become one of the first-line drugs of choice for the treatment of type 2 diabetes. In addition to diabetes treatment, GLP-1 receptor agonists are also being studied in other areas such as obesity, cardiovascular disease, and neurodegenerative diseases.

Unless otherwise specified, the term “treatment” used in this article refers to the use of a drug to alleviate or cure a diseased state, and “prevention” refers to the use of a drug to prevent the occurrence of a disease or reduce the possibility of a disease, which can be to prevent or Delay the onset or progression of these diseases.

Generally speaking, alkyl glycosides (e.g., without limitation, DDM) have been used as transmucosal absorption enhancers for polypeptides, proteins and small molecules. It was unexpected that the presence of alkyl glycosides (e.g., without limitation, DDM) improved the stability of one or more active ingredients (e.g., GLP-1 receptor agonists, and insulin, insulin analogs, and/or derivatives of insulin analogs) in various embodiments of the stable pharmaceutical compositions disclosed herein. In certain embodiments of the pharmaceutical compositions disclosed herein, the stabilities of the one or more active ingredients are sufficiently high so that the embodiments can be used for single-dose or multi-dose aqueous injectable compositions.

Pharmaceutical compositions of GLP-1 receptor agonists, especially their liquid compositions, tend to form fibrous substances during storage which makes it challenging to obtain pharmaceutical compositions having long-term stability. As shown in examples provided herein, it was unexpected that certain surfactants improved the long-term stability of GLP-1 receptor agonists in certain embodiments of the pharmaceutical composition disclosed herein. In particular, surfactants such as alkyl glycosides (e.g., without limitation, DDM) improved the stability of GLP-1 receptor agonists in various embodiments of aqueous pharmaceutical compositions.

One aspect of the invention relates to a stable pharmaceutical composition comprising a first active ingredient comprising one or more GLP-1 receptor agonists; and one or more surfactants.

In certain embodiments, the surfactants comprise alkyl glycosides (e.g., without limitation, DDM). Embodiments of the stable pharmaceutical compositions disclosed herein showed improved stability of the one or more GLP-1 receptor agonists. In certain embodiments of the stable pharmaceutical compositions disclosed herein, the presence of alkyl glycosides (e.g., without limitation, DDM) may prevent the formation of GLP-1 fibrosis when the GLP-1 receptor agonist comprises, e.g., GLP-1 or analogs or derivatives thereof, and thereby may improve physical stability of the GLP-1 receptor agonist. Alkyl glycosides (e.g., without limitation, DDM) are significantly better than other classes of surfactants.

In certain embodiments, the improved stability relates to physical stabilities. In certain embodiments, the stable pharmaceutical compositions are stable liquid pharmaceutical compositions. In certain embodiments, the stable liquid pharmaceutical compositions are stable aqueous pharmaceutical compositions. In certain embodiments, the stable aqueous pharmaceutical compositions are stable aqueous injectable pharmaceutical compositions. In certain embodiments, the stable aqueous injectable pharmaceutical compositions are single-dose or multi-dose aqueous injectable pharmaceutical compositions.

Examples of the one or more GLP-1 receptor agonists include, without limitation, GLP-1, GLP-1 analogs, and derivatives of GLP-1 analogs.

In certain embodiments, the stable pharmaceutical composition further comprises a second active ingredient comprising insulin, insulin analogs, and/or derivatives of insulin analogs, and may be referred to as a stable combination pharmaceutical composition herein. Embodiments of the stable combination pharmaceutical composition disclosed herein also have high stability of the first and the second active ingredients. In certain embodiments, the stability relates to physical stabilities. In certain embodiments, the stable combination pharmaceutical compositions are stable liquid combination pharmaceutical compositions. In certain embodiments, the stable liquid combination pharmaceutical compositions are stable aqueous combination pharmaceutical compositions. In certain embodiments, the stable aqueous combination pharmaceutical compositions are stable aqueous injectable combination pharmaceutical compositions. In certain embodiments, the stable aqueous injectable combination pharmaceutical compositions are single-dose or multi-dose aqueous injectable combination pharmaceutical compositions.

In certain embodiments of the stable combination pharmaceutical compositions disclosed herein, the second active ingredient comprises insulin or an insulin analogue. In certain embodiments of the stable combination pharmaceutical compositions disclosed herein, the insulin analogues comprise at least one of insulin lispro, insulin aspart, insulin glulisine, insulin glargine, insulin degludec, and insulin detemir. In certain embodiments of the stable combination pharmaceutical compositions disclosed herein, based on the total weight of the stable combination pharmaceutical composition, the stable combination pharmaceutical composition comprises about 0.1% wt to about 1% wt of insulin, insulin analogs, or derivatives of insulin analogs, optionally, at least about 0.1% wt to about 0.4% wt of insulin, insulin analogs, or derivatives of insulin analogs, e.g., at least about 0.2% wt to about 0.4% wt of insulin or insulin analogues. For example, the stable combination pharmaceutical composition may comprise at least one of insulin lispro, insulin aspart, insulin glulisine, insulin glargine, insulin degludec, and insulin detemir, which has a concentration of at least about 0.2%, about 0.3%, about 0.4%, about 0.5%, about 0.6%, about 0.7%, about 0.8%, about 0.9%, or about 1.0%. In certain embodiments, the stable combination pharmaceutical composition comprises insulin, insulin analogs, or derivatives of insulin analogs having a concentration of at least about 0.20% wt to about 0.40% wt, e.g., without limitation, about 0.21% wt, about 0.22% wt, about 0.23% wt, about 0.24% wt, about 0.25% wt, about 0.26% wt, about 0.27% wt, about 0.28% wt, about 0.29% wt, about 0.30% wt, about 0.310% wt, about 0.32% wt, about 0.330% wt, about 0.34% wt, about 0.35% wt, about 0.36% wt, about 0.37% wt, about 0.38% wt, about 0.39% wt, or about 0.40% wt. It should be noted that when the stable combination pharmaceutical composition is configured as a liquid pharmaceutical composition, such as a premixed water injectable composition, the above-mentioned “% wt” can also be expressed as “mg/mL.” For example, 0.40% by weight of insulin or insulin analogs or derivatives of insulin analogs means also 4.0 mg/mL insulin or insulin analogs or derivatives of insulin analogs. As shown in the examples provided in the Examples section, alkyl glycosides (e.g., without limitation, DDM) unexpectedly improved the stability of GLP-1 receptor agonists, and also simultaneously improved the stabilities of GLP-1 receptor agonists and insulin or insulin analogs or derivatives of insulin analogs. In certain embodiments, the first active ingredient comprises or is beinaglutide and the second active ingredient comprises or is insulin glargine. In certain embodiments, the first active ingredient comprises or is beinaglutide and the second active ingredient comprises or is insulin aspart.

In certain embodiments of the stable pharmaceutical compositions disclosed herein, examples of the alkyl glycosides comprises a sugar group and an alkyl group connected by a connecting group, the sugar group comprising at least one sugar selected from glucose, maltose, sucrose, or trehalose, the alkyl group comprises about 10 to about 16 carbon atoms, and the connecting group comprises at least a bond selected from glycosidic bonds, thioglycosidic bonds, or amide bonds. In certain embodiments, the alkyl group comprises about 10 to about 14 carbon atoms. In certain embodiments, the alkyl group comprises about 6 to about 20 carbon atoms. In certain embodiments, the alkyl group comprises 5, 6, 7, 8, 9, 10, 11, 12, 13, 14, 15, 16, 17, 18, 19, or 20 carbon atoms. In certain embodiments, the alkyl group comprises an unbranched alkyl and/or a branched alkyl. In certain embodiments, the alkyl group comprises a low alkyl group (e.g., C1-C8) substituted with a cycloalkyl group. Examples of the low alkyl group include, without limitation, methyl, ethyl, propyl, isopropyl, butyl, isobutyl, tert-butyl, pentyl, isopentyl, hexyl, isohexyl, heptyl, isoheptyl, octyl, isooctyl. Examples of cycloalkyl include, without limitation, cyclohexyl. In certain embodiments, the alkyl glycosides (e.g., without limitation, DDM) improve the stability of the stable pharmaceutical compositions disclosed herein. In certain embodiments, the sugar group comprises of is maltose or sucrose. In certain embodiments, the alkyl glycosides comprise at least one compound selected from n-dodecyl β-D-maltoside (DDM), sucrose monododecanoate, n-dodecyl β-D-maltoside, n-hexyl-β-D-glucoside, n-heptyl-β-D-glucoside, n-octyl-β-D-glucoside, n-nonyl-β-D-glucoside, n-decyl-β-D-glucoside, 3-cyclohexyl-1-propyl-β-D-glucoside, n-hexyl-β-D-glucopyranoside, n-octyl-β-D-maltoside, n-nonyl-β-D-maltoside, n-decyl-β-D-maltoside, cyclohexyl-methyl-β-D-maltoside, 2-cyclohexyl-ethyl-β-D-maltoside, 3-cyclohexyl-propyl-β-D-maltoside, 4-cyclohexyl-butyl-β-D-maltoside, or 5-cyclohexyl-amyl-β-D-maltoside.

In certain embodiments, based on the total weight of the stable pharmaceutical composition, the stable pharmaceutical composition comprises about 0.05% wt to about 0.50% wt of the alkyl glycosides. For example, the concentration of the alkyl glycosides is at least about 0.05% wt, about 0.06% wt, about 0.07% wt, about 0.08% wt, about 0.09% wt, about 0.10% wt, about 0.11% wt, about 0.12% wt, about 0.13% wt, about 0.14% wt, about 0.15% wt, about 0.16% wt, about 0.17% wt, about 0.18% wt, about 0.19% wt, about 0.20% wt, about 0.21% wt, about 0.22% wt, about 0.23% wt, about 0.24% wt, about 0.25% wt, about 0.26% wt, about 0.27% wt, about 0.28% wt, about 0.29% wt, about 0.30% wt, about 0.31% wt, about 0.32% wt, about 0.33% wt, about 0.34% wt, about 0.35% wt, about 0.36% wt, about 0.37% wt, about 0.38% wt, about 0.39% wt, about 0.40% wt, about 0.41% wt, about 0.42% wt, about 0.43% wt, about 0.44% wt, 0.45% wt, about 0.46% wt, about 0.47% wt, about 0.48% wt, about 0.49% wt, or about 0.50% wt. In certain embodiments, the concentration of the alkyl glycosides is at least about 0.10% wt to about 0.20% wt. In certain embodiments, the concentration of the alkyl glycosides is at least about 0.10% wt to about 0.30% wt.

In certain embodiments, the stable pharmaceutical composition is a stable combination pharmaceutical composition as disclosed herein, wherein the first active ingredient comprises one or more GLP-1 receptor agonists (e.g., GLP-1 such as beinaglutide, GLP-1 analogs, and derivatives of GLP-1 analogs), and the second active ingredient comprises or is insulin aspart, the surfactant is one or more alkyl glycosides (e.g., without limitation, DDM), and the concentration of the surfactant is about 0.05% wt to about 0.50% wt, about 0.05% wt to about 0.45% wt, about 0.05% wt to about 0.40% wt, about 0.05% wt to about 0.35% wt, about 0.05% wt to about 0.30% wt, about 0.05% wt to about 0.25% wt, 0.05% wt to about 0.20% wt, about 0.05% wt to about 0.15% wt, 0.05% wt to about 0.10% wt, 0.10% wt to about 0.50% wt, about 0.10% wt to about 0.45% wt, about 0.10% wt to about 0.40% wt, about 0.10% wt to about 0.35% wt, 0.10% wt to about 0.30% wt, about 0.10% wt to about 0.25% wt, 0.10% wt to about 0.20% wt, about 0.10% wt to about 0.15% wt, about 0.16% wt to about 0.30% wt, about 0.16% wt to about 0.25% wt, about 0.20% wt to about 0.30% wt, about 0.20% wt to about 0.25% wt, about 0.05% wt, about 0.10% wt, about 0.16% wt, about 0.20% wt, or about 0.25% wt.

In certain embodiments, the GLP-1 receptor agonists are polypeptide agonists. Optionally, the GLP-1 receptor agonists may also function as a GIP agonist. In other words, dual agonists can also be used (e.g., without limitation, P13).

In certain embodiments, the GLP-1 receptor agonists comprise at least one selected from the following: GLP-1, GLP-1 analogs or derivatives thereof, and truncated GLP-1 fragments or analogs thereof or derivatives thereof. Examples of GLP-1 analogs include, without limitation, 988927, Exendin-4 and its analogs and derivatives. In certain embodiments, the GLP-1 receptor agonists comprise at least one selected from the following: liraglutide, exenatide, lixisenatide, albiglutide, beinaglutide, doxepin laglutide, semaglutide, pepamotidide, and tilpotide. In certain embodiments, the GLP-1 receptor agonist comprises or is beinaglutide.

As used herein, the “GLP-1 receptor agonists” refer to polypeptides comprising native, extended or truncated GLP-1 polypeptides (e.g., without limitation, GLP-1(7-37)OH/NH₂, GLP-1(7-36) OH/NH₂, GLP-1(7-35)OH/NH₂), GLP-1 fragments, GLP-1 analogs and their derivatives. GLP-1 receptor agonists also may comprise dual or multiple agonists based on GLP-1 sequences, e.g., without limitation, GLP-1/GIP dual agonists (e.g., without limitation P13). As used herein, the GLP-1 compounds or the GLP-1 receptor agonists can bind to the GLP-1 receptor and initiate a signal transduction pathway that results in insulinotropic activity. GLP-1 and GLP-1 analogs may have a C-terminal free carboxyl group or a C-terminal amide group. For example, a GLP-1 analog can be a recombinant human GLP-1 (7-36) peptide with the following sequence: His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg (SEQ ID NO: 5), referred to as beinaglutide. The molecular formula of beinaglutide is C₁₄₉H₂₂₅N₃₉O₄₆, with a molecular weight of 3,298.7. beinaglutide is essentially identical to the active form of GLP-1 in vivo, except for endogenous amidation, in which the NH₂ in the native form is replaced by an OH group in the recombinant peptide. beinaglutide has a free carboxyl group at the C-terminus.

In certain embodiments of the stable pharmaceutical compositions disclosed herein, the GLP-1 receptor agonist is a polypeptide with the following sequence:

(SEQ ID NO: 1)
XHXEGTXTSDXSXXXEXXAXXXFIXWLXXGXX,

wherein,
X at position 1 is: R, or missing;
X at position 3 is: A, G, V, L, I, S, or T;
X at position 7 is: F, W, or Y;
X at position 11 is: V, S, W, I, L, K, F, or Y;
X at position 13 is: S, W, Y, F, K, I, L, or V;
X at position 14 is: Y, W, or F;
X at position 15 is: L, F, Y, or W;
X at position 17 is: G, E, D, Q, N, K, R, or C;
X at position 18 is: H, D, K, E, or Q;
X at position 20 is: A, V, I, or L;
X at position 21 is: K, R, Q, or N;
X at position 22 is: A, E, H, F, Y, W, R, I, or K;
X at position 25 is: A, E, D, S, or H;
X at position 28 is: V or I;
X at position 29 is: K, R, Q, or N;
X at position 31 is: R, R—NH2, K, or K—NH₂;
X at position 32 is: G, K, R, T, S, E, D, W, Y, F, H, or missing.

In certain embodiments of the stable pharmaceutical compositions disclosed herein, the GLP-1 receptor agonist is a polypeptide with the following sequence:

(SEQ ID NO: 2)
XXXGTXXXXXSKQXEEEAVXLXXXXLKNGGXXXXXXXXXX,

wherein:
X at position 1 is: H, R, Y, or EL;
X at position 2 is: S, G, A, or T;
X at position 3 is: D or E;
X at position 6 is: F or Y;
X at position 7 is: T, Y, or S;
X at position 8 is: S or Y;
X at position 9 is: D or E;
X at position 10 is: L or I;
X at position 14 is: L, I, V, or M;
X at position 20 is: R or K;
X at position 22 is: F or Y;
X at position 23 is: I, V, L, or M;
X at position 24 is: E or D;
X at position 25 is: W, F, or Y;
X at position 31 is: P or missing;
X at position 32 is: S or missing;
X at position 33 is: S or missing;
X at position 34 is: G or missing;
X at position 35 is: A or is missing;
X at position 36 is: P or missing;
X at position 37 is: P or missing;
X at position 38 is: P or missing;
X at position 39 is: S, R, or missing; and
X at position 40 is any amino acid or missing

In general, the stability of GLP-1 receptor agonists may be compromised by a variety of stresses. These stresses include pH, temperature, light, oscillation, and gas-liquid interface. A range of commonly used surfactants were also tested for potential stability improvement of pharmaceutical compositions comprising the first active ingredient (e.g., GLP-1 receptor agonists) and/or the second active ingredients (e.g., insulin, insulin analogs, and derivatives of insulin analogs). Examples of the surfactants tested but not effective for stability improve include, e.g., polysorbate 20, polysorbate 80, poloxamer 188, hydroxypropyl betacyclodextrin and amino acids. Provided herein are also embodiments of stable pharmaceutical compositions that do not comprise any surfactants selected from the group consisting of polysorbate 20, polysorbate 80, poloxamer 188, and hydroxypropyl betacyclodextrin. In certain embodiments, the stable pharmaceutical compositions do not comprise polysorbate 20, polysorbate 80, poloxamer 188, and hydroxypropyl betacyclodextrin or amino acids. In certain embodiments, the stable pharmaceutical compositions do not comprise surfactants other than one or more alkyl glycosides (e.g., without limitation, DDM).

In certain embodiments, the stable pharmaceutical composition is a liquid preparation. In certain embodiments, the stable pharmaceutical composition is a premixed aqueous injection preparation. In certain embodiments, the stable pharmaceutical composition is configured as a premixed preparation that is an injectable composition, preferably for subcutaneous injection.

In certain embodiments of the stable pharmaceutical compositions disclosed herein, the pharmaceutical compositions optionally comprise other pharmaceutically acceptable excipients, e.g., without limitation, buffer systems, bacteriostatic agents, pharmaceutically acceptable solubilizers, and zinc ions.

In certain embodiments, the pH of the stable pharmaceutical compositions disclosed herein is about 3.0 to about 9.0, about 3.0 to about 5.0, about 4.0 to about 5.0, or about 4.0 to about 4.5. In certain embodiments, the pH of the stable pharmaceutical compositions disclosed herein is about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, or about 9.0.

In certain embodiments, the stable pharmaceutical composition is a stable combination pharmaceutical composition as disclosed herein, wherein the first active ingredient comprises one or more GLP-1 receptor agonists (e.g., GLP-1 such as beinaglutide, GLP-1 analogs, and derivatives of GLP-1 analogs), and has a pH ranging form about 3.0 to about 4.5, about 3.1, about 3.2, about 3.3, about 3.4, about 3.5, about 3.6, about 3.7, about 3.8, about 3.9, about 4.0, about 4.1, about 4.2, about 4.3, about 4.4, or about 4.5.

In certain embodiments, the stable pharmaceutical composition is a stable combination pharmaceutical composition as disclosed herein, wherein the first active ingredient comprises one or more GLP-1 receptor agonists (e.g., GLP-1 such as beinaglutide GLP-1 analogs, and derivatives of GLP-1 analogs), and the pH is about 6.5 to about 8.5, about 6.6 to about 8.4, about 6.7 to about 8.3, about 6.8 to about 8.2, about 6.9 to about 8.2, about 7.0 to about 8.2, about 7.0 to about 8.15, about 7.0 to about 8.1, about 7.0 to about 8.0, about 7.0 to about 7.9, about 7.0 to about 7.8, about 7.0 to about 7.7, about 7.0 to about 7.6, about 7.1 to about 8.2, about 7.1 to about 8.15, about 7.1 to about 8.1, about 7.1 to about 8.0, about 7.1 to about 7.9, about 7.1 to about 7.8, about 7.1 to about 7.7, about 7.1 to about 7.6, about 7.2 to about 8.2, about 7.2 to about 8.15, about 7.2 to about 8.1, about 7.2 to about 8.0, about 7.2 to about 7.9, about 7.2 to about 7.8, about 7.2 to about 7.7, about 7.2 to about 7.6, about 7.3 to about 8.2, about 7.3 to about 8.15, about 7.3 to about 8.1, about 7.3 to about 8.0, about 7.3 to about 7.9, about 7.3 to about 7.8, about 7.3 to about 7.7, or about 7.3 to about 7.6.

In certain embodiments, the stable pharmaceutical composition is a stable combination pharmaceutical composition as disclosed herein, wherein the first active ingredient comprises one or more GLP-1 receptor agonists (e.g., GLP-1 such as beinaglutide, GLP-1 analogs, and derivatives of GLP-1 analogs), and the second active ingredient comprises or is insulin glargine, and the pH is about 3.5 to about 4.6, about 3.5 to about 4.5, about 3.5 to about 4.4, about 3.5 to about 4.3, about 3.5 to about 4.2, about 3.5 to about 4.1, about 3.5 to about 4.0, 3.6 to about 4.6, about 3.6 to about 4.5, about 3.6 to about 4.4, about 3.6 to about 4.3, about 3.6 to about 4.2, about 3.6 to about 4.1, about 3.6 to about 4.0, 3.7 to about 4.6, about 3.7 to about 4.5, about 3.7 to about 4.4, about 3.7 to about 4.3, about 3.7 to about 4.2, about 3.7 to about 4.1, about 3.7 to about 4.0, about 3.8 to about 4.6, about 3.8 to about 4.5, about 3.8 to about 4.4, about 3.8 to about 4.3, about 3.8 to about 4.2, about 3.8 to about 4.1, about 3.8 to about 4.0, about 3.9 to about 4.6, about 3.9 to about 4.5, about 3.9 to about 4.4, about 3.9 to about 4.3, about 3.9 to about 4.2, about 3.9 to about 4.1, or about 3.9 to about 4.0.

In certain embodiments, the stable pharmaceutical composition is a stable combination pharmaceutical composition as disclosed herein, wherein the first active ingredient comprises one or more GLP-1 receptor agonists (e.g., GLP-1 such as beinaglutide, GLP-1 analogs, and derivatives of GLP-1 analogs), and the second active ingredient comprises or is insulin aspart, and the pH is about 6.5 to about 8.5, about 6.6 to about 8.4, about 6.7 to about 8.3, about 6.8 to about 8.2, about 6.9 to about 8.2, about 7.0 to about 8.2, about 7.0 to about 8.15, about 7.0 to about 8.1, about 7.0 to about 8.0, about 7.0 to about 7.9, about 7.0 to about 7.8, about 7.0 to about 7.7, about 7.0 to about 7.6, about 7.1 to about 8.2, about 7.1 to about 8.15, about 7.1 to about 8.1, about 7.1 to about 8.0, about 7.1 to about 7.9, about 7.1 to about 7.8, about 7.1 to about 7.7, about 7.1 to about 7.6, about 7.2 to about 8.2, about 7.2 to about 8.15, about 7.2 to about 8.1, about 7.2 to about 8.0, about 7.2 to about 7.9, about 7.2 to about 7.8, about 7.2 to about 7.7, about 7.2 to about 7.6, about 7.3 to about 8.2, about 7.3 to about 8.15, about 7.3 to about 8.1, about 7.3 to about 8.0, about 7.3 to about 7.9, about 7.3 to about 7.8, about 7.3 to about 7.7, or about 7.3 to about 7.6.

In certain embodiments, the stable pharmaceutical composition comprises one or more buffer system commonly used for pH adjustment. Such buffer system may comprise one or more buffer salts. Examples of commonly used buffer salts include, without limitation, acetate salts, chloride salts, TRIS, HEPES, MOPS, PIPES, BES, Bis-Tris, TES, and/or phosphate salts. The concentration of the buffer salts (e.g., without limitation, acetate salts, chloride salts, and phosphate salts) can be about 0 mM to about 50 mM, about 0 mM to about 45 mM, about 0 mM to about 40 mM, about 0 mM to about 35 mM, about 0 mM to about 30 mM, about 0 mM to about 25 mM, about 0 mM to about 20 mM, about 10 mM to about 50 mM, about 10 mM to about 45 mM, about 10 mM to about 40 mM, about 10 mM to about 35 mM, about 10 mM to about 30 mM, about 10 mM to about 25 mM, about 10 mM to about 20 mM. In certain embodiments, the stable pharmaceutical composition may comprise no buffer salts.

In certain embodiments, the stable pharmaceutical composition comprises zinc ions. In certain embodiments, the concentration range of zinc ions is about 1 μg/mL to about 100 μg/mL, about 10 μg/mL to about 70 μg/mL, about 10 μg/mL to about 50 μg/mL, about 13 μg/mL to about 33 μg/mL, about 20 μg/mL to about 40 μg/mL, about 25 μg/mL to about 40 μg/mL, about 10 μg/mL, about 15 μg/mL, about 20 μg/mL, about 25 μg/mL, about 30 μg/mL, about 35 μg/mL, about 40 μg/mL, about 45 μg/mL, or about 50 μg/mL. In certain embodiments, the molar ratio of the zinc ions v. the second active ingredient (e.g., insulin, insulin analogs or derivatives of insulin analogs) is about 1:4 to about 10.8:6.

In certain embodiments, the stable pharmaceutical composition is a stable combination pharmaceutical composition as disclosed herein, wherein the first active ingredient comprises one or more GLP-1 receptor agonists (e.g., GLP-1 such as beinaglutide, GLP-1 analogs, and derivatives of GLP-1 analogs), and the second active ingredient comprises or is insulin glargine, and the stable combination pharmaceutical composition further comprises zinc ion at a concentration of about 1 μg/mL to about 100 μg/mL, about 10 μg/mL to about 70 μg/mL, about 10 μg/mL to about 50 μg/mL, about 13 μg/mL to about 33 μg/mL, about 20 μg/mL to about 40 μg/mL, about 25 μg/mL to about 40 μg/mL, about 10 μg/mL, about 15 μg/mL, about 20 μg/mL, about 25 μg/mL, about 30 μg/mL, about 35 μg/mL, about 40 μg/mL, about 45 μg/mL, or about 50 μg/mL. In certain embodiments, the molar ratio of the zinc ions v. the second active ingredient (e.g., insulin, insulin analogs or derivatives of insulin analogs) is about 1:4 to about 10.8:6.

In certain embodiments, the stable pharmaceutical composition is a stable combination pharmaceutical composition as disclosed herein, wherein the first active ingredient comprises one or more GLP-1 receptor agonists (e.g., GLP-1 such as beinaglutide, GLP-1 analogs, and derivatives of GLP-1 analogs), and the second active ingredient comprises or is insulin aspart, and the stable combination pharmaceutical composition further comprises zinc ion at a concentration of about 1 μg/mL to about 100 μg/mL, about 10 μg/mL to about 70 μg/mL, about 10 μg/mL to about 50 μg/mL, about 13 μg/mL to about 33 μg/mL, about 20 μg/mL to about 40 μg/mL, about 25 μg/mL to about 40 μg/mL, about 10 μg/mL, about 15 μg/mL, about 20 μg/mL, about 25 μg/mL, about 30 μg/mL, about 35 μg/mL, about 40 μg/mL, about 45 μg/mL, or about 50 μg/mL. In certain embodiments, the molar ratio of the zinc ions v. the second active ingredient (e.g., insulin, insulin analogs or derivatives of insulin analogs) is about 1:4 to about 10.8:6.

In certain embodiments, the stable pharmaceutical composition may comprise one or more types of anion ions. Examples of the anion ions include, without limitation, chloride ions, thiocyanate ions (KSCN), para-aminobenzoate ions (PABA), and 1-hydroxy-3-nitrobenzoate ions (4H3N). In certain embodiments, the concentration of the anion ions (e.g., chloride ion or other examples disclosed herein) is at least about 10 mM, at least about 20 mM, at least about 30 mM, at least about 40 mM, at least about 50 mM, at least about 60 mM, at least about 70 mM, at least about 80 mM, at least about 90 mM, or at least about 100 mM.

In certain embodiments, the stable pharmaceutical composition is a stable combination pharmaceutical composition as disclosed herein, wherein the first active ingredient comprises one or more GLP-1 receptor agonists (e.g., GLP-1 such as beinaglutide, GLP-1 analogs, and derivatives of GLP-1 analogs), and the second active ingredient comprises or is insulin aspart or insulin glargine, and the stable combination pharmaceutical composition further comprises anion ions, e.g., without limitation, Cl⁻, KSCN⁻, PABA⁻, and 4H3N⁻. In certain embodiments, the concentration of the anion ions (e.g., chloride ion or other examples disclosed herein) is at least about 10 mM, at least about 20 mM, at least about 30 mM, at least about 40 mM, at least about 50 mM, at least about 60 mM, at least about 70 mM, at least about 80 mM, at least about 90 mM, or at least about 100 mM.

In certain embodiments, the stable pharmaceutical composition comprises one or more solubilizers. The term “solvent” used in this article refers to a solvent that assists or enhances the dissolving ability that can improve solubility. The term “solvent” used herein does not include surfactant. In certain embodiments, examples of solubilizers are, without limitation, glycerin, mannitol, propylene glycol, or combinations thereof. Based on the total volume of the stable pharmaceutical compositions, the solubilizer concentration can be about 0 mg/mL to about 40 mg/mL, about 0 mg/mL to about 50 mg/mL, about 10 mg/mL to about 50 mg/mL, about 15 mg/mL, about 20 mg/mL, about 25 mg/mL, about 30 mg/mL, about 35 mg/mL, about 40 mg/mL, or about 45 mg/mL. In certain embodiments, the solubilizer is glycerin. Based on the total volume of the stable pharmaceutical composition, in certain embodiments, the solubilizer is glycerin or propylene glycol, having a concentration of about 0 mg/mL to about 40 mg/mL, about 10 mg/mL to about 25 mg/ml, about 12 mg/mL, about 14 mg/mL, about 16 mg/mL, about 17 mg/mL, about 18 mg/mL, about 20 mg/mL, about 22 mg/mL, or about 24 mg/mL. Based on the total volume of the stable pharmaceutical composition, in certain embodiments, the solubilizer is mannitol having a concentration of about 0 mg/mL to about 50 mg/mL, 0 mg/mL to about 40 mg/mL, about 10 mg/mL to about 25 mg/ml, about 12 mg/mL, about 14 mg/mL, about 16 mg/mL, about 17 mg/mL, about 18 mg/mL, about 20 mg/mL, about 22 mg/mL, or about 24 mg/mL.

In certain embodiments, the stable pharmaceutical composition comprises one or more osmotic pressure regulators, e.g., without limitation, glycerin, mannitol, propylene glycol, or combinations thereof.

In certain embodiments, the stable pharmaceutical compositions are isotonic systems to plasma in subjects, which usually has osmotic pressure is about 280 mOsm/L to about 320 mOsm/L. In certain embodiments, the stable pharmaceutical composition has an osmotic pressure of about 280 mOsm/L to about 320 mOsm/L, about 270 mOsm/L to about 330 mOsm/L, or about 260 mOsm/L to about 340 mOsm/L. In certain embodiments, the pharmaceutical composition comprises 40 mg/mL mannitol and 5 mg/mL propylene glycol as osmotic pressure regulators. In certain embodiments, the stable pharmaceutical composition is an insulin glargine injection composition or insulin glargine/GLP-1 combination pharmaceutical composition, the osmotic pressure regulator is glycerin. The glycerin concentration in the insulin glargine injection composition is 17 mg/ml, and the glycerin concentration in the combination pharmaceutical composition is 20 mg/ml. Other osmotic pressure regulators that may be suitable include, without limitation, mannitol, glycerol, propylene glycol, and combinations thereof.

In certain embodiments, the stable pharmaceutical composition comprises one or more bacteriostatic agents. Optionally, the bacteriostatic agent includes at least one compound selected form phenolic compounds, aromatic alcohol compounds, phenoxyethanol and chlorohydrin compounds. In certain embodiments, the bacteriostatic agent includes at least one compound selected from phenol, m-cresol, benzyl alcohol, phenoxyethanol, or chlorobutanol. In certain embodiments, the bacteriostatic agent is phenol. In certain embodiments, the stable pharmaceutical composition comprises phenol at a concentration of about 2.0 mg/mL to about 4.0 mg/mL, about 2.0 mg/mL, about 2.1 mg/mL, about 2.2 mg/mL, about 2.3 mg/mL, about 2.4 mg/mL, about 2.5 mg/mL, about 2.6 mg/mL, about 2.7 mg/mL, about 2.8 mg/mL, about 2.9 mg/mL, about 3.0 mg/mL, about 3.1 mg/mL, about 3.2 mg/mL, about 3.3 mg/mL, about 3.4 mg/mL, about 3.5 mg/mL, about 3.6 mg/mL, about 3.7 mg/mL, about 3.8 mg/mL, about 3.9 mg/mL, about 4.0 mg/mL, about 2.10 mg/mL, about 2.11 mg/mL, about 2.12 mg/mL, about 2.13 mg/mL, about 2.14 mg/mL, about 2.15 mg/mL, about 2.16 mg/mL, about 2.17 mg/mL, about 2.18 mg/mL, about 2.19 mg/mL, about 2.20 mg/mL, about 2.21 mg/mL, about 2.22 mg/mL, about 2.23 mg/mL, about 2.24 mg/mL, about 2.25 mg/mL, about 2.26 mg/mL, about 2.27 mg/mL, about 2.28 mg/mL, about 2.29 mg/mL, about 2.30 mg/mL, about 2.31 mg/mL, about 2.32 mg/mL, about 2.33 mg/mL, about 2.34 mg/mL, about 2.35 mg/mL, about 2.36 mg/mL, about 2.37 mg/mL, about 2.38 mg/mL, about 2.39 mg/mL, or about 2.40 mg/mL.

In certain embodiments, the stable pharmaceutical composition may comprise a GLP-1 receptor agonist at a concentration of about 0.1 mg/mL to about 10 mg/mL, about 0.1 mg/mL to about 2.5 mg/mL, about 0.1 mg/mL to about 1 mg/mL, about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1.0 mg/mL. about 1.1 mg/mL, about 1.2 mg/mL, about 1.3 mg/mL, about 1.4 mg/mL, about 1.5 mg/mL, about 1.6 mg/mL, about 1.7 mg/mL, about 1.8 mg/mL, about 1.9 mg/mL, about 2.0 mg/mL, about 2.1 mg/mL, about 2.2 mg/mL, about 2.3 mg/mL, about 2.4 mg/mL, about 2.5 mg/mL, about 2.6 mg/mL, about 2.7 mg/mL, about 2.8 mg/mL, about 2.9 mg/mL, about 3.0 mg/mL. about 3.1 mg/mL, about 3.2 mg/mL, about 3.3 mg/mL, about 3.4 mg/mL, about 3.5 mg/mL, about 3.6 mg/mL, about 3.7 mg/mL, about 3.8 mg/mL, about 3.9 mg/mL, or about 4.0 mg/mL.

In certain embodiments of the stable pharmaceutical compositions disclosed herein, no formation of foreign object is observed in an accelerated stability test at 25° C. for 3 months (25C3M), and the peptide % (i.e., the amount of peptide before the test over the amount of peptide after the test) is not less than about 93%, or not less than about 94%. Examples of the corresponding methods are described in the General Methods in the Examples section.

Another aspect of the invention relates to a method for improving the storage stability of a pharmaceutical composition comprising a first active ingredient comprising one or more GLP-1 receptor agonists. In certain embodiments of the method disclosed herein, the method comprises mixing one or more surfactants (e.g., without limitation, alkyl glycosides (e.g., without limitation, DDM)) with the pharmaceutical composition. In certain embodiments, the pharmaceutical composition is a combination pharmaceutical composition further comprising a second active ingredient comprising insulin, insulin analogs, and/or derivatives of insulin analogs. Examples of the insulin analogs include, without limitation, insulin lispro, insulin aspart, insulin glulisine, insulin glargine, insulin degludec, and insulin detemir.

In certain embodiments, the one or more alkyl glycosides comprise at least one of DDM, sucrose monododecanoate, n-dodecyl β-D-maltoside, n-hexyl-β-D-glucoside, n-heptyl-β-D-glucoside, n-octyl-β-D-glucoside, n-nonyl-β-D-glucoside, n-decyl-β-D-glucoside, 3-cyclohexyl-1-propyl-β-D-glucoside, n-hexyl-β-D-glucopyranoside, n-octyl-β-D-maltoside, n-nonyl-β-D-maltoside, n-decyl-β-D-maltoside, cyclohexyl-methyl-β-D-maltoside, 2-cyclohexyl-ethyl-β-D-maltoside, 3-cyclohexyl-propyl-β-D-maltoside, 4-cyclohexyl-butyl-β-D-maltoside, or 5-cyclohexyl-amyl-β-D-maltoside. In certain embodiments, the alkyl glycoside is DDM.

Another aspect of the invention relates to the use of the stable pharmaceutical composition for the manufacture of a medicament for use in: (i) preventing and/or treating diabetes, and/or for reducing HbA1C; (ii) delaying or preventing the progression of diabetes, delaying the progression of impaired glucose tolerance to insulin-requiring type 2 diabetes, delaying or preventing insulin resistance, and/or delaying the progression of type 2 diabetes that does not require insulin to insulin-requiring type 2 diabetes; (iii) improving β-cell function, and/or restoring β-cell glucose sensitivity; (iv) preventing and/or treating eating disorders; reducing gastric motility; delaying gastric emptying; (v) preventing and/or treating complications of obesity disease; (vi) preventing and/or treating complications of diabetes; and/or (vii) preventing and/or treating cardiovascular disease. In certain embodiments, the stable pharmaceutical composition is a stable combination pharmaceutical composition further comprising a second active ingredient comprising insulin, insulin analogs, and/or derivatives of insulin analogs.

Another aspect of the invention relates to the stable pharmaceutical composition for use in: (i) the prevention and/or treatment of diabetes, and/or for the reduction of HbA1C; (ii) the delay or prevention of the progression of diabetes, the delay of the progression of impaired glucose tolerance to insulin-requiring type 2 diabetes, the delay or prevention of insulin resistance, and/or the delay of the progression of type 2 diabetes that does not require insulin to insulin-requiring type 2 diabetes; (iii) the improvement of 3-cell function, and/or the restoration of 3-cell glucose sensitivity; (iv) the prevention and/or treatment of eating disorders; the reduction of gastric motility; the delay of gastric emptying; (v) the prevention and/or treatment of complications of obesity disease; (vi) the prevention and/or treatment of complications of diabetes; and/or (vii) the prevention and/or treatment of cardiovascular disease. In certain embodiments, the stable pharmaceutical composition is a stable combination pharmaceutical composition further comprising a second active ingredient comprising insulin, insulin analogs, and/or derivatives of insulin analogs.

Another aspect of the invention relates to methods for (i) preventing and/or treating diabetes, and/or for reducing HbA1C; (ii) delaying or preventing the progression of diabetes, delaying the progression of impaired glucose tolerance to insulin-requiring type 2 diabetes, delaying or preventing insulin resistance, and/or delaying the progression of type 2 diabetes that does not require insulin to insulin-requiring type 2 diabetes; (iii) improving β-cell function, and/or restoring β-cell glucose sensitivity; (iv) preventing and/or treating eating disorders; reducing gastric motility; delaying gastric emptying; (v) preventing and/or treating complications of obesity disease; (vi) preventing and/or treating complications of diabetes; and/or (vii) preventing and/or treating cardiovascular disease in a subject comprising administering to the subject a therapeutically effective amount of the stable pharmaceutical composition. In certain embodiments, the stable pharmaceutical composition is a stable combination pharmaceutical composition further comprising a second active ingredient comprising insulin, insulin analogs, and/or derivatives of insulin analogs.

The following examples are provided to better illustrate the claimed invention and the embodiments described herein, but these examples should not be construed to limit the scope of the invention. Unless otherwise specified, the specific materials listed in the following examples are for illustrative purposes only and are not intended to limit the present disclosure. It will be apparent to those skilled in the art that various equivalents, changes and modifications can be made without departing from the scope of the invention, and it is understood that such equivalent embodiments are intended to be included herein. Furthermore, all references cited in this application are incorporated by reference in their entirety as if fully set forth herein.

### EXAMPLES

### General Methods

Unless otherwise specified, the methods described in the General Methods section were used in the Examples described in the Examples section.

### I. ThT Test

ThT test was a common method for monitoring protein fibril formation. Thioflavin T (ThT) was a benzothiazole fluorescent dye that highly specifically bound to amyloid fibrils and led to enhanced fluorescence after binding. This test used changes in fluorescence value to measure the kinetic process of protein fibril formation. Fibril formation happened when the fluorescence value exceeded a predetermined threshold. Unless otherwise specified, in the embodiment of the present disclosure, the predetermined threshold was three times of the initial fluorescence value of the measured point, i.e., Tn/T1=3.

### ThT Test Protocol:

1. Sample preparation (according to the ThT test sample prescription list): mixing the test sample (500 μL) with thioflavin T (aq., 1 mM, 25 L) to provide a first mixture, adding 200 μL of the first mixture to a black V-shaped 96-well plate (Eppendorf), and setting 2 replicate wells for each sample (each sample respectively applied to 2 wells for measurement).
2. Sample incubation: Covering the 96-well plate with sealing tape and sealing it to prevent sample evaporation and contamination; placing the 96-well plate in a mixer (Thermo Mixer C, Eppendorf), placing it away from light at 37° C. and a rotating speed of 300 rpm for incubation, the 96-well plate used was 96 well Costar blk/clrbtm (a 96-well plate produced by Costar for laboratory use, comprising a black bottom and a transparent top), although other suitable 96-well plate can also be used.
3. Fluorescence measurement parameters: setting the fluorescence measurement mode on a M5e multifunctional microplate reader (Molecular Devices, LLC), the excitation wavelength set to 430 nm, the emission wavelength set to 470 nm, and the measurement temperature set to room temperature (around 25° C.), and the 96-well plate being 96 well Costar blk/clrbtm.
4. Sample measurement: Incubating the test sample to a set time for fluorescence measurement, and taking the average of the fluorescence measurement values of 2 replicate wells for data analysis. The initial fluorescence value was the fluorescence measurement value after incubation at 37° C. for 1 hour. The 96-well plate was wrapped with aluminum foil for protection from light after the 96-well plate was taken out.

### II. Stability Test

There are two parts of stability investigation: appearance inspection and peptide amount inspection. Appearance inspection checked the stability by visually observing the clarity of the test composition and any visible foreign object formation. Peptide amount inspection measured the peptide amount by HPLC to inspect the peptide stability of the composition. Compositions passed the appearance inspection were selected and subjected to accelerated testing in a 25° C. stability test chamber, and peptide amount inspection was conducted at inspection time points.

### 1. Appearance Inspection

Samples were prepared according to the requirements of the stability test prescription table, and stored in a stability test chamber at 25° C. for accelerated testing. Samples were taken out at various time points (e.g., 14 days, 1 month, 2 months, 3 months, etc.) and subjected to appearance inspection. Appearance inspection observed indicators such as the clarity of the composition, visible foreign object formation, etc., through visual inspection. A composition met the requirements if no obvious abnormality was observed in the appearance inspection, and the composition was subjected to the next step of HPLC testing. Otherwise, the composition did not meet the requirements and would not be subjected to the next step of measurement.

### 2. Peptide Amount Inspection

The compositions used for stability test were stored in a stability test box at 25° C. for accelerated testing. At different time points (e.g., 14 days, 1 month, 2 months, 3 months, etc.), the compositions that passed the appearance inspection were inspected for peptide amount. 100 μL of the composition was taken as a test sample. The amounts of GLP-1 and insulin glargine in the composition was measured using HPLC to evaluate the stability.

The chromatographic column used for HPLC was: Thermo BioBasic 18 column (250×4.6 mm, 5 μm), the detection wavelength was 214 nm, and the column temperature was 35° C.; the mobile phase A was: phosphate (pH 2.5)-sodium chloride-25% acetonitrile, the mobile phase B was: phosphate (pH 2.5)-sodium chloride-65% acetonitrile; and a gradient elution program was used, in which the concentration of phase B gradually increased from 4% to 17% within 20 min, and then further increased to 53% within 18 min for gradient elution.

Under the same chromatographic condition, a chromatogram having insulin glargine and GLP-1 as separate single component could be obtained.

The peaks of insulin glargine and GLP-1 or their derivatives could be assigned by comparing the chromatogram of the combination composition with the chromatograms of single-component compositions of insulin glargine and GLP-1, respectively. Generally speaking, insulin glargine and/or its derivatives had relatively weak retention on the C18 chromatographic column, and the peak position was in the first half of the combination pharmaceutical composition chromatogram. GLP-1 and/or its derivative, beinaglutide, had relatively strong retention, and their peak position was in the second half of the pharmaceutical composition chromatogram. The GLP-1 peptide amount remained (herein also referred to as “GLP-1 peptide %” or “peptide %” unless specified otherwise) can be obtained by calculating the ratio of the peak area of GLP-1 at various stability inspection time points to the initial value. In other words, at different stability inspection time points, take composition samples for HPLC analysis to obtain the peak area corresponding to the chromatographic peak of GLP-1. Then compare the GLP-1 peak area at each stability inspection time point with the GLP-1 peak area at the initial time point (to) to obtain the percentage of GLP-1 peptide remained. The same method can be used to calculate insulin glargine peptide % as well.

### Example 1

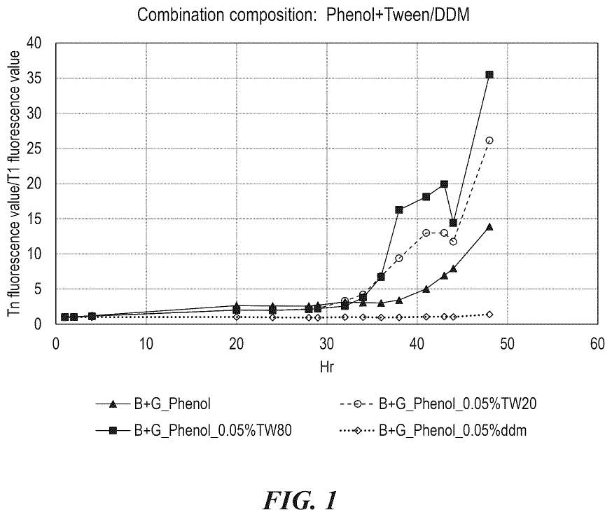

A series of pharmaceutical compositions comprising GLP-1 receptor agonists (Compositions 1 to 28) were prepared with various components as set forth in Table 1, zinc acetate was used as the source of zinc ion. ThT tests were conducted on these compositions respectively, and showed that DDM significantly inhibited the formation of GLP-1 fibrosis and improved the physical stability of the pharmaceutical compositions. Results are discussed with reference to FIGS. 1 to 8 respectively.

TABLE 1
Compositions 1 to 28.

| | | | Insulin | | | | | |
|---|---|---|---|---|---|---|---|---|
| Composition | Composition | Beinaglutide | glargine | | Bacteriostatic | | | |
| No. | description | (B) | (G) | Glycerin | agent | Zinc ions, | pH | Surfactant |
| 1 | B + G_phenol | 1 mg/mL | 3.64 mg/mL | 20 mg/mL | Phenol 2.2 | 30 μg/mL | 4.0 | / |
| | | | | | mg/mL | | | |
| 2 | B + G_ | 1 mg/mL | 3.64 mg/mL | 20 mg/mL | Phenol 2.2 | 30 μg/mL | 4.0 | 0.05%Tween |
| | phenol_ | | | | mg/mL | | | 20 |
| | 0.05%TW20 | | | | | | | |
| 3 | B + G_ | 1 mg/mL | 3.64 mg/mL | 20 mg/mL | Phenol 2.2 | 30 μg/mL | 4.0 | 0.05%Tween |
| | phenol_ | | | | mg/mL | | | 80 |
| | 0.05%TW80 | | | | | | | |
| 4 | B + G_ | 1 mg/mL | 3.64 mg/mL | 20 mg/mL | Phenol 2.2 | 30 μg/mL | 4.0 | 0.05% DDM |
| | phenol_ | | | | mg/mL | | | |
| | 0.05%DDM | | | | | | | |
| 5 | GLP1_ | 1 mg/mL | / | 20 mg/mL | Phenol 2.2 | 30 μg/mL | 4.0 | / |
| | phenol | | | | mg/mL | | | |
| 6 | GLP1_ | 1 mg/mL | / | 20 mg/mL | Phenol 2.2 | 30 μg/mL | 4.0 | 0.05%Tween |
| | phenol_ | | | | mg/mL | | | 20 |
| | 0.05%TW20 | | | | | | | |
| 7 | GLP1_ | 1 mg/mL | / | 20 mg/mL | Phenol 2.2 | 30 μg/mL | 4.0 | 0.05%Tween |
| | phenol_ | | | | mg/mL | | | 80 |
| | 0.05%TW80 | | | | | | | |
| 8 | GLP1_ | 1 mg/mL | / | 20 mg/mL | Phenol 2.2 | 30 μg/mL | 4.0 | 0.05% DDM |
| | phenol_ | | | | mg/mL | | | |
| | 0.05% | | | | | | | |
| | DDM | | | | | | | |
| 9 | BNLT Lot1 | Beinaglutide, 2 mg/mL; mannitol30 mg/mL; propylene glycol 5 mg/mL; | | | | | | / |
| | (New) | phenol 2.2 mg/mL; acetate 10 mM; pH4.0 | | | | | | |
| 10 | BNLT Lot2 | Beinaglutide, 2 mg/mL; mannitol30 mg/mL; propylene glycol 5 mg/mL; | | | | | | 0.01% DDM |
| | +0.01% | phenol 2.2mg/mL; acetate 10 mM; pH4.0 | | | | | | |
| | DDM | | | | | | | |
| 11 | BNLT Lot2 | Beinaglutide, 2 mg/mL; mannitol30 mg/mL; propylene glycol 5 mg/mL; | | | | | | 0.05% DDM |
| | +0.05%DDM | phenol 2.2 mg/mL; acetate 10 mM; pH4.0 | | | | | | |
| 12 | BNLT Lot2 | Beinaglutide, 2 mg/mL; mannitol30 mg/mL; propylene glycol 5 mg/mL; | | | | | | 0.1% DDM |
| | +0.1% DDM | phenol 2.2 mg/mL; acetate 10 mM; pH4.0 | | | | | | |
| 13 | B + G_phenol_ | 1 mg/mL | 3.64 mg/mL | 20 mg/mL | Phenol | 30 μg/mL | 4.0 | 0.05% DDM |
| | 0.05% | | | | | | | |
| | DDM | | | | | | | |
| 14 | B + G_m- | 1 mg/mL | 3.64 mg/mL | 20 mg/mL | m-Cresol | 30 μg/mL | 4.0 | 0.05% DDM |
| | cresol | | | | | | | |
| | 0.05% | | | | | | | |
| | DDM | | | | | | | |
| 15 | B + G_benzyl | 1 mg/mL | 3.64 mg/mL | 20 mg/mL | Benzyl alcohol | 30 μg/mL | 4.0 | 0.05% DDM |
| | alcohol_0.05% | | | | | | | |
| | DDM | | | | | | | |
| 16 | B + G_Trichloro- | 1 mg/mL | 3.64 mg/mL | 20 mg/mL | Trichloro- | 30 μg/mL | 4.0 | 0.05% DDM |
| | butanol_ | | | | butanol | | | |
| | 0.05% DDM | | | | | | | |
| 17 | B + G_Pheno- | 1 mg/mL | 3.64 mg/mL | 20 mg/mL | Phenoxyethanol | 30 μg/mL | 4.0 | 0.05% DDM |
| | xyethano_ | | | | | | | |
| | 0.05% | | | | | | | |
| | DDM | | | | | | | |
| | | | | Osmotic | | | | |
| Composition | Composition | | Insulin | pressure | Bacteriostatic | | | |
| No. | Lot No. | GLP1 | glargine | regulator | agent | Zn | PH | DDM |
| 18 | 20201112-1 | 1 mg/mL | 3.64 mg/mL | Glycerin 20 | Phenol 2.2 | 30 μg/mL | 4.0 | 0.1% DDM |
| | | | | mg/mL | mg/mL | | | |
| 19 | 20201112-2 | 0.5 mg/mL | 3.64 mg/mL | Glycerin 20 | Phenol 2.2 | 30 μg/mL | 4.0 | 0.1% DDM |
| | | | | mg/mL | mg/mL | | | |
| 20 | 20201112-3 | 1 mg/mL | 3.64 mg/mL | Mannitol | Phenol 2.2 | 30 μg/mL | 3.6- | / |
| | | | | 2 mg/mL, | mg/mL | | 3.7 | |
| | | | | glycerin 20 | | | | |
| | | | | mg/mL | | | | |
| 21 | 20201112-4 | 1 mg/mL | 3.64 mg/mL | Mannitol | Phenol 2.2 | 30 μg/mL | 3.6- | / |
| | | | | 5 mg/mL, | mg/mL | | 3.7 | |
| | | | | propylene | | | | |
| | | | | glycol 14 | | | | |
| | | | | mg/mL | | | | |
| 22 | 20201112-5 | 0.5 mg/mL | 3.64 mg/mL | Mannitol | Phenol 2.2 | 30 μg/mL | 3.8 | / |
| | | | | 40 mg/mL, | mg/mL | | | |
| | | | | propylene | | | | |
| | | | | glycol 2 | | | | |
| | | | | mg/ml | | | | |
| Composition | Composition | | | | | | | |

| No. | description | Peptide sequence | Peptide concentration/Buffer system | pH | Surfactant |
|---|---|---|---|---|---|
| 23 | GLP-1 | HAEGTFTSDVSSYLE | Active ingredient peptide concentration: 1 | 4.0 | / |
| | | GQAAKEFIAWLVKG | mg/mL; | | |
| 24 | GLP1-DDM | R (SEQ ID NO: 5) | Buffer system: 10 mM HAc-NaAc | 4.0 | 0.1% DDM |
| 25 | P13 | ELAEGTFTSDLSKQM | | 4.0 | / |
| | | EEEAVRLFIEWLKNG | | | |
| 26 | P13-DDM | GPSSGAPPPS-NH2 | | 4.0 | 0.1% DDM |
| | | (SEQ ID NO: 3) | | | |
| 27 | 988927 | HGEGTFTSDSSSYLE | | 4.0 | / |
| | | EQAAKEFIAWLVKG | | | |
| 28 | 988927- | RG (SEQ ID NO: 4) | | 4.0 | 0.1% DDM |
| | DDM | | | | |

### Summary of ThT Test Results on Compositions 1-28

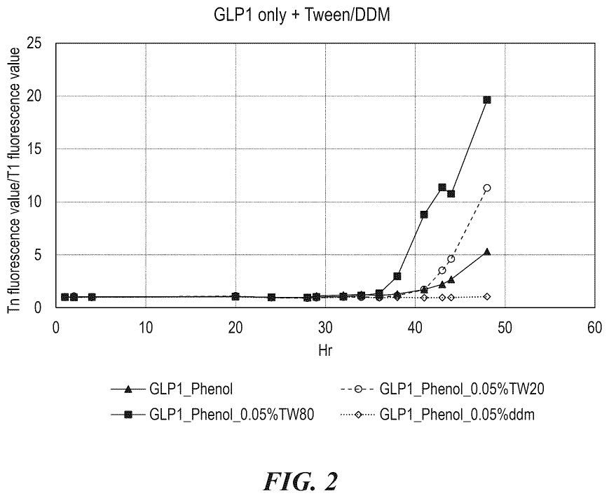

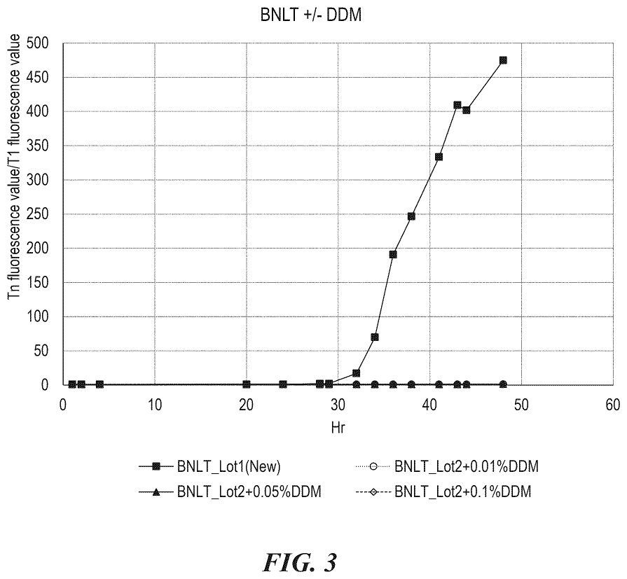

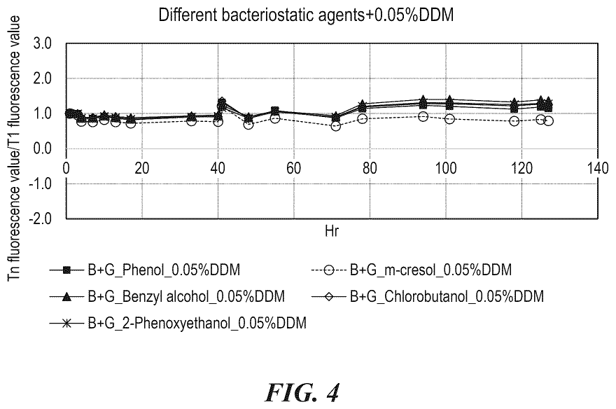

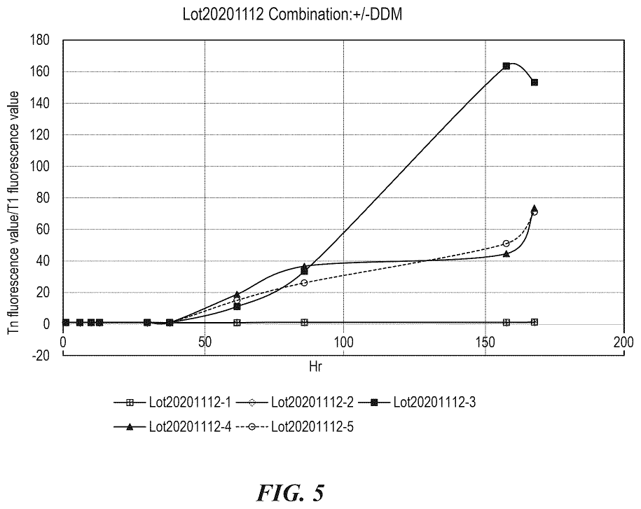

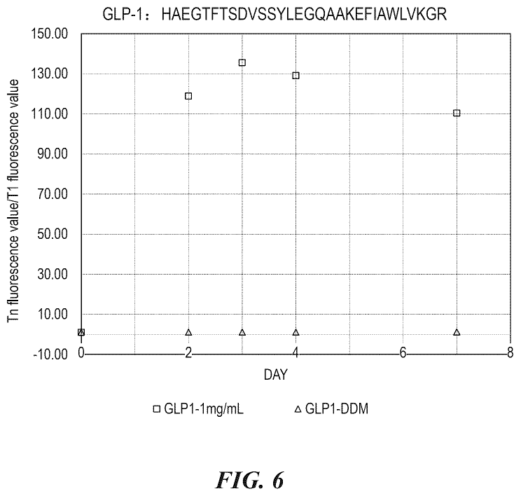

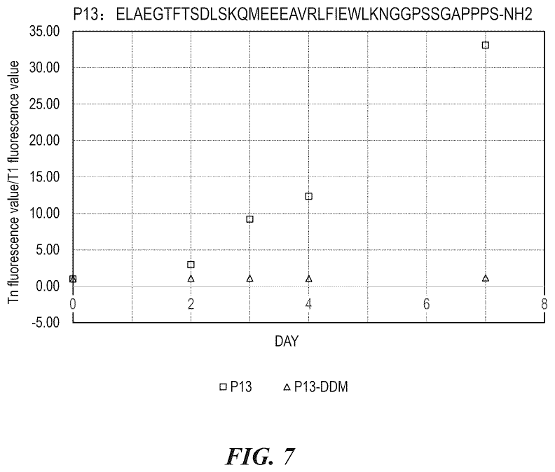

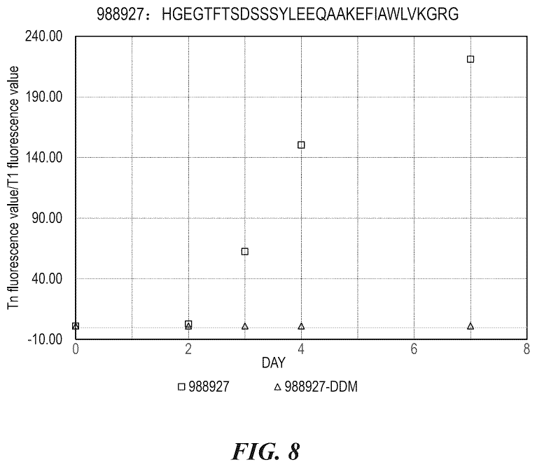

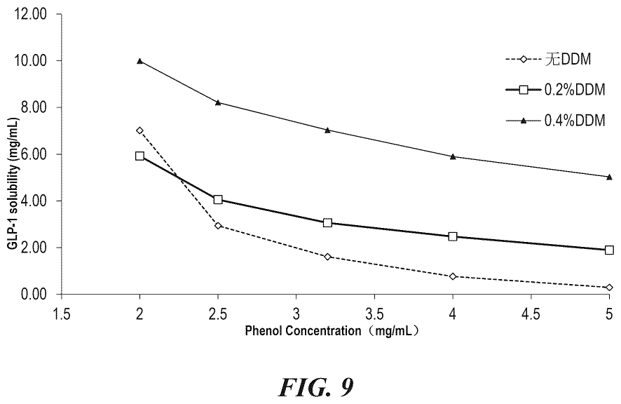

1. The ThT test results of Compositions 1 to 4 (FIG. 1) showed that for the insulin glargine/beinaglutide stock solution combination composition test samples, DDM inhibited the formation of GLP-1 fibrosis and improved physical stability significantly better than other surfactants tested (e.g., Tween 20 and Tween 80). As shown in FIG. 1, the fluorescence increase was not obvious before 30 hours (early stage) and therefore, no obvious tendency to form fibrils was observed. As the incubation time extended beyond 30 hours, an obvious increase in fluorescence value was observed in Composition 1 that did not comprise surfactant (solid triangle, B+G_phenol), as well as Compositions 2 and 3 that comprised Tween (hollow circle, B+G_phenol_0.05% TW20) and Tween 80 (solid square, B+G_phenol_0.05% TW80), respectively, but no significant increase of fluorescence value in Composition 4 which comprised DDM (hollow diamond, B+G_phenol_0.05% DDM). The fluorescence values of Compositions 1-3 continued increasing rapidly over time, which indicates that significant protein fiber formation occurred in. However, in the test sample comprising DDM, the fluorescence value did not change significantly close to 50 hours. Therefore, DDM was shown to be significantly better than other surfactants tested in inhibiting the formation of GLP-1 fibrosis and improving physical stability in the insulin glargine/beinaglutide stock solution combination composition test samples.
2. The ThT test results of Compositions 5 to 8 (FIG. 2) showed that for the beinaglutide stock solution test samples, DDM inhibited the formation of GLP-1 fibrosis and improved physical stability significantly better than other surfactants (e.g., Tween 20 and Tween 80). As shown in FIG. 2, the fluorescence increase was not obvious before 30 hours (early stage) and therefore, no obvious tendency to form fibrils was observed. As the incubation time extended beyond 30 hours, an obvious increase in fluorescence value was observed in Composition 5 that did not comprise surfactant (solid triangle, GLP-1_phenol), as well as Compositions 6 and 7 that comprised Tween 20 (hollow circle, GLP-1_phenol_0.05% TW20) and Tween 80 (solid square, GLP-1_phenol_0.05% TW80), respectively, but no significant increase of fluorescence value in Composition 8 which comprised DDM (hollow diamond, GLP-1_phenol_0.05% DDM). The fluorescence values of Compositions 5-7 continued increasing rapidly over time, which indicates that significant protein fiber formation occurred in. However, in the test sample comprising DDM, the fluorescence value did not change significantly close to 50 hours. Therefore, DDM was shown to be significantly better than other surfactants tested in inhibiting the formation of GLP-1 fibrosis and improving physical stability in beinaglutide stock solution test sample.
3. The ThT test results of Compositions 9 to 12 (FIG. 3) showed that for the beinaglutide composition test samples, DDM inhibited the formation of GLP-1 fibrosis and improved physical stability significantly. As shown in FIG. 3, the fluorescence increase was not obvious before 30 hours (early stage) and therefore, no obvious tendency to form fibrils was observed. As the incubation time extended beyond 30 hours, an obvious increase in fluorescence value was observed in Composition 9 that did not comprise surfactant (solid square, BNLT_Lot1(New)), while in compositions that comprised DDM, no significant increase of fluorescence value was observed, even for Composition 10 with a DDM concentration as low as 0.01% (hollow circle, BNLT_Lot2+0.01% DDM), see also Composition 11 with a DDM concentration of 0.05% (solid triangle, BNLT_Lot2+0.05% DDM) and Composition 12 with a DDM concentration of 0.1% (hollow diamond, BNLT_Lot2+0.1% DDM). In the test sample comprising DDM, the fluorescence value did not change significantly close to 50 hours. Therefore, DDM was shown to significantly inhibit the formation of GLP-1 fibrosis and improving physical stability in beinaglutide composition test sample.
4. The ThT test results of Compositions 13 to 17 (FIG. 4) showed that for the composition comprising various bacteriostatic agents, DDM (0.05%) significantly inhibited the formation of protein fibrosis and improved physical stability. As shown in FIG. 4, the fluorescence increase was not obvious for the whole time tested (>150 hr) in insulin glargine/beinaglutide stock solution combination composition test samples with various bacteriostatic agents: Composition 13 with phenol (solid square, B+G_phenol+0.05% DDM), Composition 14 with m-cresol (hollow circle, B+G_m-cresol+0.05% DDM), Composition 15 with benzyl alcohol (solid triangle, B+G_benzyl alcohol+0.05% DDM), Composition 16 with trichlorobutanol (hollow diamond, B+G_trichlorobutanol+0.05% DDM), and Composition 17 with phenoxyethanol (checked square, B+G_phenoxyethanol+0.05% DDM). Therefore, DDM was shown to significantly inhibit the formation of GLP-1 fibrosis and improving physical stability in compositions with various bacteriostatic agents.
5. The ThT test results of Compositions 18 to 22 (FIG. 5) showed that compared to conventional compositions without DDM, composition comprising DDM significantly inhibited the formation of protein fibrosis and improved physical stability. As shown in FIG. 5, the fluorescence increase significantly in Composition 20 (solid square, 20201112-3), Composition 21 (solid triangle, 20201112-4), and Composition 22 (hollow circle, 20201112-5) which were conventional compositions without DDM. However, no significant fluorescence change was observed in Composition 18 (checked square, 20201112-1) and Composition 19 (hollow diamond, 20201112-2) which comprised DDM. Therefore, DDM was shown to have advantage over conventional compositions without DDM on inhibition of protein fibrosis formation and physical stability.
6. The ThT test results of Compositions 23 to 28 (FIGS. 6-8) showed that DDM significantly inhibited the formation of GLP-1 fibrosis and improved physical stability in compositions comprising GLP-1, GLP-1 analog (e.g., 988927), and GLP-1 based GLP-1/GIP dual agonist (e.g., P13). As shown in FIG. 6, the fluorescence increase significantly after incubation at 37° C. for 1 to 2 days in Composition 23 (FIG. 6, hollow square, GLP1-1 mg/mL), Composition 25 (FIG. 7, hollow square, P13), and Composition 27 (FIG. 8, hollow square, 988927), which did not comprise DDM. After 7 days of incubation, the fluorescence value of Compositions 23 and 27 increased more than 100 times of the respective initial values, and the fluorescence value of Composition 25 increased more than 35 times of the initial value. However, no significant fluorescence change was observed in compositions with 0.01% DDM through the 1-week of test time: Composition 24 (FIG. 6, hollow triangle, GLP1-DDM), Composition 26 (FIG. 7, hollow triangle, P13-DDM), and Composition 28 (FIG. 8, hollow triangle, 9889270DDM). Therefore, DDM was shown to significantly inhibit GLP-1 fibrosis formation and improve physical stability in compositions comprising GLP-1, GLP-1 analog (e.g., 988927), and GLP-1 based GLP-1/GIP dual agonist (e.g., P13).
7. DDM improved GLP-1 solubility in the presence of phenol at neutral pH. GLP-1 solubility was evaluated over phenol concentration at neutral pH, without DDM, with 0.2% DDM, and with 0.4% DDM. GLP-1 solubility reduced with increased phenol concentration (FIG. 9). However, presence of DDM improved GLP-1 solubility compared to GLP-1 composition without DDM (diamond, without DDM), and the higher DDM concentration (triangle, 0.4% DDM) showed further improvement of GLP-1 solubility than the lower DDM concentration (square, 0.2% DDM).

### Example 2

A series of combination compositions comprising GLP-1 receptor agonists and insulin glargine were prepared, and their stability tests showed that DDM improved the stability of the compositions.

Compositions A1 to A20 having the components listed in Table 2A were prepared, in which zinc chloride was used as the source of zinc ions. Both appearance inspection and peptide amount inspection were conducted to determine the stabilities.

TABLE 2A
Compositions A1 to A20.

| Composition | | | Insulin | | Bacteriostatic | | | |
|---|---|---|---|---|---|---|---|---|
| No. | Composition Lot No. | Beinaglutide | glargine | Glycerin | agent | Zn ion | pH | Surfactant |
| A1 | Lot20200519 | 1 mg/mL | 3.64 mg/mL | 20 mg/mL | Phenol 2.2 | 30 μg/mL | 4.0 | / |
| | | | | | mg/mL | | | |
| A2 | Lot20200713/BEM040 + | 1 mg/mL | 3.64 mg/mL | 20 mg/mL | Phenol 2.2 | 30 μg/mL | 4.0 | 0.1% Tween 20 |
| | GLP1 1.0_TW20 | | | | mg/mL | | | |
| A3 | Lot20200713/BEM040 + | 1 mg/mL | 3.64 mg/mL | 20 mg/mL | Phenol 2.2 | 30 μg/mL | 4.0 | 0.1% Tween 80 |
| | GLP1 1.0_TW80 | | | | mg/mL | | | |
| A4 | Lot20200928_4# | 1 mg/mL | 3.64 mg/mL | 20 mg/mL | Phenol 2.2 | 30 μg/mL | 4.0 | 0.1% DDM |
| | | | | | mg/mL | | | |
| | | | | Osmotic | | | | |
| Composition | Composition | | Insulin | pressure | Bacteriostatic | | | |
| No. | Lot No. | Beinaglutide | glargine | regulator | agent | Zn | pH | DDM |
| A5 | 20201112-1 | 1 mg/mL | 3.64 mg/mL | Glycerin | Phenol 2.2 | 30 μg/mL | 4.0 | 0.10% |
| | | | | 20 mg/mL | mg/mL | | | |
| A6 | 20201112-2 | 0.5 mg/mL | 3.64 mg/mL | Glycerin | Phenol 2.2 | 30 μg/mL | 4.0 | 0.10% |
| | | | | 20 mg/mL | mg/mL | | | |
| A7 | 20201112-3 | 1 mg/mL | 3.64 mg/mL | Mannitol | Phenol 2.2 | 30 μg/mL | 3.7 | / |
| | | | | 2 mg/mL, | mg/mL | | | |
| | | | | glycerin | | | | |
| | | | | 20 mg/mL | | | | |
| A8 | 20201112-4 | 1 mg/mL | 3.64 mg/mL | Mannitol | Phenol 2.2 | 30 μg/mL | 3.7 | / |
| | | | | 5 mg/mL, | mg/mL | | | |
| | | | | propylene | | | | |
| | | | | glycol | | | | |
| | | | | 14 mg/mL | | | | |
| A9 | 20201112-5 | 0.5 mg/mL | 3.64 mg/mL | Mannitol | Phenol 2.2 | 30 μg/mL | 3.8 | / |
| | | | | 40 mg/mL, | mg/mL | | | |
| | | | | propylene | | | | |
| | | | | glycol | | | | |
| | | | | 2 mg/mL | | | | |
| A10 | Lot20201014_1# | 1 mg/mL | 3.64 mg/mL | 20 mg/mL | Phenol 2.2 | 30 μg/mL | 4.0 | 0.10% |
| | | | | | mg/mL | | | |
| A11 | Lot20201014_2# | 1 mg/mL | 3.64 mg/mL | 20 mg/mL | 2.2 mg/mL | 30 μg/mL | 4.0 | 0.10% |
| A12 | Lot20201014_3# | 1 mg/mL | 3.64 mg/mL | 20 mg/mL | Benzyl alcohol | 30 μg/mL | 4.0 | 0.10% |
| | | | | | 2.2 mg/mL | | | |
| A13 | Lot20201014_4# | 1 mg/mL | 3.64 mg/mL | 20 mg/mL | Trichlorobutanol | 30 μg/mL | 4.0 | 0.10% |
| | | | | | 2.2 mg/mL | | | |
| A14 | Lot20201014_5# | 1 mg/mL | 3.64 mg/mL | 20 mg/mL | Phenoxyethanol | 30 μg/mL | 4.0 | 0.10% |
| | | | | | 2.2 mg/mL | | | |
| Composition | Composition | | Insulin | | Bacteriostatic | | | |
| No. | Lot No. | Beinaglutide | glargine | Glycerin | agent | [symbol] | pH | DDM |
| A15 | Lot20200928-3# | 1 mg/mL | 3.64 mg/mL | 20 mg/mL | Phenol 2.2 | 30 μg/mL | 4.0 | 0.05% |
| | | | | | mg/mL | | | |
| A16 | Lot20200928-4# | 1 mg/mL | 3.64 mg/mL | 20 mg/mL | Phenol 2.2 | 30 μg/mL | 4.0 | 0.10% |
| | | | | | mg/mL | | | |
| A17 | Lot20200928-5# | 1 mg/mL | 3.64 mg/mL | 20 mg/mL | Phenol 2.2 | 30 μg/mL | 4.0 | 0.15% |
| | | | | | mg/mL | | | |
| A18 | Lot20200928-6# | 1 mg/mL | 3.64 mg/mL | 20 mg/mL | Phenol 2.2 | 30 μg/mL | 4.0 | 0.20% |
| | | | | | mg/mL | | | |
| A19 | Lot20200928-11# | 1 mg/mL | 3.64 mg/mL | 20 mg/mL | Phenol 2.2 | 30 μg/mL | 4.2 | 0.10% |
| | | | | | mg/mL | | | |
| A20 | Lot20200928-12# | 1 mg/mL | 3.64 mg/mL | 20 mg/mL | Phenol 2.2 | 30 μg/mL | 4.4 | 0.10% |
| | | | | | mg/mL | | | |

### Summary of Stability Test Results

1. The stability test results of Compositions A1˜A4 are summarized in Table 2B. The data show that DDM significantly improved the stability of the compositions compared to other surfactants, e.g., Tween 20 and Tween 80.

TABLE 2B
Stability test results of insulin glargine/ beinaglutide stock solution combination composition

| Composition | | | Appearance | 25C14D¹- | 25C1M²- | 25C2M³- | 25C3M⁴- |
|---|---|---|---|---|---|---|---|
| No. | Lot No. | Notes | inspection | peptide %* | peptide % | peptide % | peptide % |
| A1 | Lot20200519 | Without | / | 96.46 | 93.36 | / | / |
| | | surfactants | | | | | |
| A2 | Lot20200713/BEM040 + | Combination | / | 96.10 | 93.84 | / | / |
| | GLP1 1.0_TW20 | composition | | | | | |
| | | comprising | | | | | |
| | | 0.1% Tween 20 | | | | | |
| A3 | Lot20200713/BEM040 + | Combination | 25C14D | / | / | / | / |
| | GLP1 1.0_TW80 | composition | Appearance | | | | |
| | | comprising | inspection | | | | |
| | | 0.1% Tween 80 | failed (gel | | | | |
| | | | formation) | | | | |
| A4 | Lot20200928_4# | Combination | Y | 99.16 | 97.90 | 96.08 | 94.66 |
| | | composition | | | | | |
| | | comprising | | | | | |
| | | 0.1% DDM | | | | | |

Y: Appearance inspection passed.
*Peptide % means the percentage of GLP-1 peptide amount of a composition after storage at the specified condition compared to the GLP-1 amount before storage.
¹25C14D means storage at 25° C. for 14 days.
²25C1M means storage at 25° C. for 1 month.
³25C2M means storage at 25° C. for 2 months.
⁴25C3M means storage at 25° C. for 3 months.

As shown in Table 2B, after one month of accelerated testing at 25° C. (25C1M), the peptide %0 of the composition without surfactant (Composition A1, Lot20200519) reduced to 93.6%, and the peptide % of the composition with Tween 20 (Composition A2: Lot20200713/BEM040+GLP1 1.0_TW20) reduced to 93.84%. After 14 days of accelerated testing at 25° C. (25C1D), the composition comprising Tween 80 (Composition A3: Lot20200713/BEM040+GLP1 1.0_TW80) showed gel formation and failed the appearance inspection. Composition A4, which comprised 0.1% DDM preparation (Composition A4: Lot20200928_4 #) showed a peptide %0 of 94.6600 after three month of accelerated testing at 25° C. (2503M), and passed the appearance inspection. Therefore, the experimental results showed that compared to Tween 20 and Tween 80, DDM significantly increased the stability of the composition.
2. The stability test results of Compositions A5˜A9 are summarized in Table 2C. The data show that DDM significantly improved the stability of the compositions compared to conventional excipients.

TABLE 2C
Stability test results of insulin glargine/ beinaglutide stock solution combination composition

| Composition | Composition | Composition | Appearance | 25C14D- | 25C1M- | 25C2M- | 25C3M- |
|---|---|---|---|---|---|---|---|
| No. | Lot No. | description | inspection | peptide %* | peptide % | peptide % | peptide % |
| A7 | 20201112-3 | Conventional | Y | 98.22 | 95.96 | 93.01 | 90.89 |
| | | excipients | | | | | |
| A8 | 20201112-4 | Conventional | Y | 98.40 | 96.22 | 93.19 | 90.90 |
| | | excipients | | | | | |
| A9 | 20201112-5 | GLP1, 0.5, | Y | 98.67 | 96.87 | 93.65 | 91.21 |
| | | conventional | | | | | |
| | | excipients | | | | | |
| A5 | 20201112_1 | Comprising | Y | 99.29 | 97.67 | 96.22 | 94.51 |
| | | 0.1% DDM | | | | | |
| A6 | 20201112_2 | GLP1, 0.5, | Y | 99.65 | 97.97 | 96.12 | 94.06 |
| | | comprising | | | | | |
| | | 0.1% DDM | | | | | |

Y: Appearance inspection passed.
*Peptide % means the percentage of GLP-1 amount after storage at the specified condition over the GLP-1 amount before storage.

As shown in Table 2C, Compositions A7 to A9 had various combinations of glycerol, mannitol, and propylene glycol but without DDM. Lowering the pH of compositions may be beneficial to improve the physical stability and avoiding abnormal appearance. However, when the pH was lowered, the peptide % of Compositions A7 to A9 reduced to about 90% after 3 months in the accelerated stability test (25C3M). However, the combination compositions comprising DDM (Compositions A5-A6) showed accelerated stability in three months at 25° C., with a peptide % greater than 94%. Therefore, compared to the conventional excipients, DDM significantly improved the stability of the compositions.
3. The stability test results of Compositions A10˜A14 are summarized in Table 2D. The data show that DDM significantly improved the stability of the compositions with various with bacteriostatic agents.

TABLE 2D
Stability test results of insulin glargine/ beinaglutide stock solution-
DDM combination composition comprising different bacteriostatic agents.

| Composition | Composition | Composition | Appearance | 25C14D- | 25C1M- | 25C2M- | 25C3M- |
|---|---|---|---|---|---|---|---|
| No. | Lot No. | Description | inspection | peptide %* | peptide % | peptide % | peptide % |
| A10 | Lot20201014_1# | Bacteriostatic | Y | 99.66 | 98.38 | 95.97 | 93.61 |
| | | agent: Phenol | | | | | |
| A11 | Lot20201014_2# | Bacteriostatic | Y | 99.70 | 97.93 | 95.09 | 92.10 |
| | | agent: m-Cresol | | | | | |
| A12 | Lot20201014_3# | Bacteriostatic | Y | 98.79 | 97.70 | 95.14 | 93.05 |
| | | agent: Benzyl | | | | | |
| | | alcohol | | | | | |
| A13 | Lot20201014_4# | Bacteriostatic | Y | 99.47 | 98.04 | 96.07 | 93.57 |
| | | agent: | | | | | |
| | | Trichlorobutanol | | | | | |
| A14 | Lot20201014_5# | Bacteriostatic | Y | 99.87 | 98.34 | 95.99 | 94.08 |
| | | agent: | | | | | |
| | | Phenoxyethanol | | | | | |

Y: Appearance inspection passed.
*Peptide % means the percentage of GLP-1 amount after storage at the specified condition over the GLP-1 amount before storage.

Bacteriostatic agents tested are commonly used in injection-grade compositions, e.g., phenol, m-cresol, benzyl alcohol, trichlorobutanol, and phenoxyethanol. All compositions tested compared DDM and passed the accelerated stability test at 25° C. for 3 months with a peptide %0 greater than 9200. Therefore, DDM not only significantly improved the stability of the compositions, but was also compatible with various bacteriostatic agents commonly used in injections.
4. The stability test results of Compositions A15-A18 are summarized in Table 2E. Compositions with various DDM concentrations were compared.

TABLE 2E
Stability test results of insulin glargine/ beinaglutide stock solution
combination compositions comprising different concentrations of DDM.

| Composition | Composition | Composition | GLP-1 Peptide %* | | | |
|---|---|---|---|---|---|---|
| No. | Lot No. | Description | 25C14D | 25C28D | 25C60D | 25C90D |
| A15 | Lot20200928-3# | 0.05% DDM, pH 4.0 | 99.06 | 97.66 | / | / |
| A16 | Lot20200928-4# | 0.1% DDM, pH 4.0 | 99.16 | 97.90 | 96.08 | 94.66 |
| A17 | Lot20200928-5# | 0.15% DDM, pH 4.0 | 98.40 | 97.21 | 95.10 | 93.68 |
| A18 | Lot20200928-6# | 0.2% DDM, pH 4.0 | 98.91 | 97.87 | 95.79 | 94.60 |

*Peptide % means the percentage of peptide amount after storage at the specified condition over the peptide amount before storage.

The accelerated stability test at 25° C. for 2 months (25C60D) of Composition A15 (0.05% DDM) failed the visual inspection, while compositions comprising 0.1% DDM or higher all passed appearance inspection after the accelerated stability test at 25° C. for 3 months (25C90D), with GLP-1 peptide % greater than 93%. Therefore, a DDM concentration of or above 0.1% improved the physical stability of compositions to a greater extent than a DDM concentration of about 0.05%.
5. The stability test results of Compositions A16, A19 and A20 are summarized in Table 2E. DDM-comprising compositions with different pH were compared.

TABLE 2F
Stability investigation results of insulin glargine/ beinaglutide stock
solution-DDM combination compositions with different pH values

| Composition | Composition | Composition | GLP-1 peptide %* | | | |
|---|---|---|---|---|---|---|
| No. | Lot No. | Description | 25C14D | 25C28D | 25C60D | 25C90D |
| A16 | Lot20200928-4# | 0.1% DDM, pH 4.0 | 99.16 | 97.90 | 96.08 | 94.66 |
| A19 | Lot20200928-11# | 0.1% DDM, pH 4.2 | 99.31 | 97.97 | 95.79 | 93.68 |
| A20 | Lot20200928-12# | 0.1% DDM, pH 4.4 | 99.15 | 95.49 | 95.55 | 93.89 |

*Peptide % means the percentage of peptide amount after storage at the specified condition over the peptide amount before storage.

As shown in Table 2F, within the pH range of 4.0-4.5, the combination compositions comprising 0.1% DDM did not fail the appearance inspection in the accelerated stability test at 25° C. for three months, and the peptide % was greater than 93%. Therefore, DDM effectively improved the stability of preparations in the pH range of 4.0˜4.5.
6. A series of combination compositions comprising GLP-1 receptor agonists and insulin glargine were prepared (Compositions A30-41), and their stability were tested.

TABLE 2G
Compositions A30-

| Composition | Composition | | Insulin | | Bacteriostatic | | | |
|---|---|---|---|---|---|---|---|---|
| No. | Lot No. | Beinaglutide | glargine | Solubilizers | agent | Zn ion | pH/Buffer | DDM |
| A30 | Lot20210512-1 | 1 mg/mL | 3.64 mg/mL | Glycerin 20 | Phenol 2.2 | 30 μg/mL | 3.8/AcOH | 0.1% |
| | | | | mg/mL | mg/mL | | | |
| A31 | Lot20200928-4 | 1 mg/mL | 3.64 mg/mL | Glycerin 20 | Phenol 2.2 | 30 μg/mL | 4.0/ | 0.1% |
| | | | | mg/mL | mg/mL | | AcOH—HCl | |
| A32 | Lot20200928-11 | 1 mg/mL | 3.64 mg/mL | Glycerin 20 | Phenol 2.2 | 30 μg/mL | 4.2/ | 0.1% |
| | | | | mg/mL | mg/mL | | AcOH—HCl | |
| A33 | Lot20200928-12 | 1 mg/mL | 3.64 mg/mL | Glycerin 20 | Phenol 2.2 | 30 μg/mL | 4.4/ | 0.1% |
| | | | | mg/mL | mg/mL | | AcOH—HCl | |
| A34 | Lot20220928-1 | 1 mg/mL | 3.64 mg/mL | Glycerin 20 | Phenol 2.2 | 30 μg/mL | 3.8/HCl | / |
| | | | | mg/mL, | mg/mL | | | |
| | | | | mannitol 2 | | | | |
| | | | | mg/mL | | | | |
| A35 | Lot20220928-2 | 1 mg/mL | 3.64 mg/mL | Glycerin 2 | Phenol 2.2 | 30 μg/mL | 3.8/HCl | / |
| | | | | mg/mL, | mg/mL | | | |
| | | | | mannitol 40 | | | | |
| | | | | mg/mL | | | | |
| A36 | Lot20220928-3 | 1 mg/mL | 3.64 mg/mL | Glycerin 15 | Phenol 2.2 | 30 μg/mL | 3.8/HCl | / |
| | | | | mg/mL, | mg/mL | | | |
| | | | | mannitol 25 | | | | |
| | | | | mg/mL | | | | |
| A37 | Lot20220928-4 | 1 mg/mL | 3.64 mg/mL | propylene | Phenol 2.2 | 30 μg/mL | 3.8/HCl | / |
| | | | | glycol 2 | mg/mL | | | |
| | | | | mg/mL, | | | | |
| | | | | mannitol 35 | | | | |
| | | | | mg/mL | | | | |
| A38 | Lot20220928-5 | 1 mg/mL | 3.64 mg/mL | Glycerin 20 | Phenol 2.2 | 30 μg/mL | 3.8/HCl | 0.1% DDM |
| | | | | mg/mL, | mg/mL | | | |
| | | | | mannitol 2 | | | | |
| | | | | mg/mL | | | | |
| A39 | Lot20220928-6 | 1 mg/mL | 3.64 mg/mL | Glycerin 2 | Phenol 2.2 | 30 μg/mL | 3.8/HCl | 0.1% DDM |
| | | | | mg/mL, | mg/mL | | | |
| | | | | mannitol 40 | | | | |
| | | | | mg/mL | | | | |
| A40 | Lot20220928-7 | 1 mg/mL | 3.64 mg/mL | Glycerin 15 | Phenol 2.2 | 30 μg/mL | 3.8/HCl | 0.1% DDM |
| | | | | mg/mL, | mg/mL | | | |
| | | | | mannitol 25 | | | | |
| | | | | mg/mL | | | | |
| A41 | Lot20220928-8 | 1 mg/mL | 3.64 mg/mL | Propylene | Phenol 2.2 | 30 μg/mL | 3.8/HCl | 0.1% DDM |
| | | | | glycol 2 | mg/mL | | | |
| | | | | mg/mL, | | | | |
| | | | | mannitol 35 | | | | |
| | | | | mg/mL | | | | |

As shown in Table 2H, in the pH range of 3.8 to 4.4, the combination compositions comprising 0.1% DDM showed insulin glargine peptide % greater than 97.23% and GLP-1 peptide % greater than 95.49% after the accelerated stability test at 25° C. for three months. Therefore, DDM effectively improved the stability of preparations in the pH range of 3.8 to 4.4.

TABLE 2H
Stability investigation results of insulin glargine/ beinaglutide-
DDM combination compositions with different pH values

| Composition | Composition | | GLP1 peptide % 25 C. | | | | Insulin glargine peptide % 25 C. | | | |
|---|---|---|---|---|---|---|---|---|---|---|
| No. | Lot No. | pH | 14 D | 28 D | 2 M | 3 M | 14 D | 28 D | 2 M | 3 M |
| A30 | Lot20210512-1 | 3.8 | 99.21 | 97.91 | 96.72 | 94.67 | 99.45 | 98.77 | 99.23 | 98.39 |
| A31 | Lot20200928-4 | 4.0 | 99.16 | 97.90 | 96.08 | 94.66 | 99.77 | 99.19 | 99.59 | 98.79 |
| A32 | Lot20200928-11 | 4.2 | 99.31 | 97.97 | 95.79 | 93.68 | 99.61 | 98.94 | 99.03 | 98.07 |
| A33 | Lot20200928-12 | 4.4 | 99.15 | 95.49 | 95.55 | 93.89 | 98.26 | 97.62 | 97.23 | 97.56 |

As shown in Table 2I, in the pH range of 3.8 to 4.4, the combination compositions comprising 0.1% DDM showed insulin glargine peptide %0 greater than 97.23% and GLP-1 peptide % greater than 95.49% after the accelerated stability test at 25° C. for three months.

TABLE 2I
Stability investigation results of insulin glargine/beinaglutide-
DDM combination compositions with or without DDM

| | | Appearance inspection | | GLP-1 peptide % | |
|---|---|---|---|---|---|
| Composition No. | Composition Lot No. | 40 C. 14 D | 40 C. 28 D | 40 C. 14 D | 40 C. 28 D |
| A34 | Lot20220928-1 | Gel | Gel | / | / |
| A35 | Lot20220928-2 | Gel | Gel | / | / |
| A36 | Lot20220928-3 | Ok | Gel at the bottom | / | / |
| A37 | Lot20220928-4 | Slight gel | Gel at the bottom | / | / |
| A38 | Lot20220928-5 | Good | Good | 93.12% | 87.39% |
| A39 | Lot20220928-6 | Good | Good | 93.97% | 88.52% |
| A40 | Lot20220928-7 | Good | Good | 94.42% | 88.77% |
| A41 | Lot20220928-8 | Good | Good | 94.25% | 88.62% |

Combination compositions comprising different solubilizer combinations (glycerin/mannitol/propylene glycol) were tested with or without DDM. The test combination compositions without DDM could not pass the appearance inspection after the 40C28D accelerated test conditions. The presence of 0.1% DDM in the combination compositions improved the appearance after the 40C28D accelerated test, and the GLP-1 peptide %0 after the accelerated test of 40C14D and 40C28D was greater than 93% and 87%, respectively.

### Example 3

A series of combination compositions comprising GLP-1 receptor agonists and insulin aspart were prepared, and their stability tests showed that DDM improved the stability of the compositions. Unless specified otherwise, the buffer system of the combination compositions of Example 3 was PB.

Compositions B1 to B20 having the components listed in Table 3A were prepared. Both appearance inspection and peptide amount inspection were conducted to determine the stabilities.

TABLE 3A
Compositions B1 to B20.

| Composition | Composition | | Insulin | | Bacteriostatic | | | | |
|---|---|---|---|---|---|---|---|---|---|
| No. | Lot No. | Beinaglutide | aspart | Glycerin | agent | Zn ion | pH | Surfactant | NaCl |
| B1 | Lot20230807-1 | 1 mg/mL | 3.5 mg/mL | 20 mg/mL | Phenol 3.2 | 20 μg/mL | 7.6 | 0.2% DDM | 0 mM |
| | | | | | mg/mL | | | | |
| B2 | Lot20230807-2 | 1 mg/mL | 3.5 mg/mL | 20 mg/mL | Phenol 3.2 | 20 μg/mL | 7.6 | 0.2% DDM | 10 mM |
| | | | | | mg/mL | | | | |
| B3 | Lot20230807-3 | 1 mg/mL | 3.5 mg/mL | 20 mg/mL | Phenol 3.2 | 20 μg/mL | 7.6 | 0.2% DDM | 20 mM |
| | | | | | mg/mL | | | | |
| B4 | Lot20230807-4 | 1 mg/mL | 3.5 mg/mL | 20 mg/mL | Phenol 3.2 | 20 μg/mL | 7.6 | 0.2% DDM | 50 mM |
| | | | | | mg/mL | | | | |
| B5 | Lot20230807-5 | 1 mg/mL | 3.5 mg/mL | 20 mg/mL | Phenol 3.2 | 20 μg/mL | 7.6 | 0.2% DDM | 100 mM |
| | | | | | mg/mL | | | | |
| B6 | Lot20240123-3 | 1 mg/mL | 3.5 mg/mL | 20 mg/mL | Phenol 3.2 | 20 μg/mL | 7.6 | 0.16% DDM | 40 mM |
| | | | | | mg/mL | | | | |
| B7 | Lot20240109-2 | 1 mg/mL | 3.5 mg/mL | 20 mg/mL | Phenol 3.2 | 20 μg/mL | 7.6 | 0.2% DDM | 40 mM |
| | | | | | mg/mL | | | | |
| B8 | Lot20240109-3 | 1 mg/mL | 3.5 mg/mL | 20 mg/mL | Phenol 3.2 | 20 μg/mL | 7.6 | 0.25% DDM | 40 mM |
| | | | | | mg/mL | | | | |
| B9 | Lot20221110-1 | 1 mg/mL | 3.5 mg/mL | 20 mg/mL | Phenol 2.2 | 25 μg/mL | 8.15 | 0.2% DDM | |
| | | | | | mg/mL | | | | |
| B10 | Lot20221025-2 | 1 mg/mL | 3.5 mg/mL | 20 mg/mL | Phenol 1.2 | 25 μg/mL | 7.6 | 0.2% DDM | |
| | | | | | mg/mL, | | | | |
| | | | | | m-cresol | | | | |
| | | | | | 1.376 mg/mL | | | | |
| B11 | Lot20231018-1 | 1 mg/mL | 3.5 mg/mL | 20 mg/mL | Phenol 3.2 | 20 μg/mL | 7.6 | 0.2% DDM | 30 mM |
| | | | | | mg/mL | | | | |
| B12 | Lot20231018-3 | 1 mg/mL | 3.5 mg/mL | 20 mg/mL | Phenol 3.2 | 20 μg/mL | 7.4 | 0.2% DDM | 30 mM |
| | | | | | mg/mL | | | | |
| B13 | Lot20230608-J4 | 1 mg/mL | 3.5 mg/mL | 20 mg/mL | Phenol 3.2 | 1 μg/mL + | 7.6 | 0.2% DDM | |
| | | | | | mg/mL | EDTA 0.3 μM | | | |
| B14 | Lot20230511-3 | 1 mg/mL | 3.5 mg/mL | 20 mg/mL | Phenol 3.2 | 10 μg/mL | 7.6 | 0.2% DDM | |
| | | | | | mg/mL | | | | |
| B15 | Lot20230511-4 | 1 mg/mL | 3.5 mg/mL | 20 mg/mL | Phenol 3.2 | 20 μg/mL | 7.6 | 0.2% DDM | |
| | | | | | mg/mL | | | | |
| B16 | Lot20230511-5 | 1 mg/mL | 3.5 mg/mL | 20 mg/mL | Phenol 3.2 | 25 μg/mL | 7.6 | 0.2% DDM | |
| | | | | | mg/mL | | | | |

1. Compositions B1-B5: Embodiment of the Combination Pharmaceutical Composition of Insulin Aspart and GLP-1 (Beinaglutide) with Different NaCl Concentrations.

DLS (dynamic light scattering) light intensity spectrum was used to show the size of particles present in Compositions B1-B5.

The combination pharmaceutical compositions without NaCl (Composition B1) had predominantly large GLP-1 particles above 100 nm after accelerated test for 14 days at 40° C. (40C14D), which indicated that GLP-1 had significant protein aggregation.

The combination pharmaceutical compositions with NaCl concentration of 10 mM (Composition B2) had significantly reduced amount of large particles above 100 nm.

No large particles above 100 nm were observed for the combination pharmaceutical compositions with NaCl concentrations of 20 mM (Composition B3) or above (Compositions B4-B5). Most GLP-1 had a particle size below 10 nm.

Stability test results of Compositions B1-B5 are summarized in Table 3B below.

TABLE 3B
Stability test results of insulin aspart/GLP-1 combination composition

| | | | Appearance | 40 C. 14 D peptide % | |
|---|---|---|---|---|---|
| Composition No. | Lot No. | Notes | inspection | Insulin aspart | GLP-1 |
| B1 | Lot20230807-1 | NaCl 0 mM | Failed | / | / |
| B2 | Lot20230807-2 | NaCl 10 mM | Passed | 90.8 | 93.22 |
| B3 | Lot20230807-3 | NaCl 20 mM | Passed | 91.39 | 91.72 |
| B4 | Lot20230807-4 | NaCl 50 mM | Passed | 92.19 | 91.67 |
| B5 | Lot20230807-5 | NaCl 100 mM | Passed | 92.51 | 90.51 |

Combination compositions without NaCl failed the appearance inspection after the 40C14D accelerated test. As the NaCl concentration increased to above 10 mM, the appearance of the combination compositions improved significantly during the 40C14D accelerated test; after which, the insulin aspart peptide % remained above 90.8%, and the GLP-1 peptide % was above 90.5%.

2. Compositions B6-B8: Embodiment of the Combination Pharmaceutical Composition of Insulin Aspart and GLP-1 (Beinaglutide) with Different DDM Concentrations.

After accelerated test at 40° C. for 2 months (40C2M), Composition B6 (0.16% DDM) failed the appearance inspection and showed white flocculent precipitate.

Composition B8 (0.25% DDM) failed the appearance inspection after accelerated test at 40° C. for 14 days (40C14D), although the same composition passed the appearance inspection after storage at 4° C. for 3 months (4C3M).

Composition B7 (0.2% DDM) passed the appearance inspection after both accelerated test at 40° C. for 14 days (40C14D), and after storage at 4° C. for 3 months (4C3M).

3. Compositions B9-B12: Embodiment of the Combination Pharmaceutical Composition of Insulin Aspart and GLP-1 (Beinaglutide) with Different pH (pH 7.4, 7.6 and 8.15).

Stability test results of Compositions B1-B5 are summarized in Table 3B below.

TABLE 3B
Stability test results of insulin aspart/GLP-1
combination composition with different pH

| | | | 40C2W peptide % | | 40C4W peptide % | |
|---|---|---|---|---|---|---|
| | | | Insulin | | Insulin | |
| Composition No. | Lot No. | Notes | aspart | GLP-1 | aspart | GLP-1 |
| B9 | Lot20221110-1 | pH 8.1 | 86.09% | 95.29% | / | / |
| B10 | Lot20221025-2 | pH 7.6 | 92.14% | 96.44% | 85.94% | 93.93% |
| B11 | Lot20231018-1 | pH 7.6 | 95.41% | 95.95% | / | / |
| B12 | Lot20231018-3 | pH 7.4 | 94.44% | 94.50% | / | / |

Combination composition with pH 8.15 (Composition B9) maintained appearance stability for 3 months at 4° C. However, the peptide % data showed that insulin aspart had lower peptide % after 40C2W accelerated test in Composition B9 (pH 8.15) than Composition B10 (pH 7.6).

Composition B11 (pH 7.6) and Composition B12 (pH 7.4) both passed appearance inspection after 40C2W accelerated test, although after 40C4W accelerated test, both composition maintained good fluidity with foreign object.

4. Compositions B13-B16: Embodiment of the Combination Pharmaceutical Composition of Insulin Aspart and GLP-1 (Beinaglutide) with Different Zinc Ion Concentration (1 μg/mL, 10 μg/mL, 20 μg/mL, and 25 μg/mL).

Stability test results of Compositions B1-B5 are summarized in Table 3C below.

TABLE 3C
Stability test results of insulin aspart/GLP-1 combination
composition with different zinc concentration

| Composition | | | | Appearance |
|---|---|---|---|---|
| No. | Lot No. | Notes | Appearance inspection 25 C. | inspection 40 C. |
| B13 | Lot20230608- | Zn 1 μg/mL + | 6 D turbidity 1.72; 25 C. 3 M | 4 D turbidity |
| | J4 | EDTA | turbidity 2.11, good appearance | 4.811/4.16 |
| B14 | Lot20230511- | Zn 10 | turbidity 3.57; 4 Wprecipitation | 2 Wprecipitation |
| | 3 | μg/mL | | |
| B15 | Lot20230511- | Zn 20 | 4 W slightly muddy, turbidity 5.03 | 2 W turbidity |
| | 4 | μg/mL | | 9.37/7.42 |
| B16 | Lot20230511- | Zn 25 | 2 Wprecipitation; 4 W slightly | 2 W turbidity |
| | 5 | μg/mL | muddy, turbidity 4.25 | 9.63/8.71 |

Composition B13 comprised EDTA that could complex all zinc in the solution, therefore, Composition B13 could be considered as not comprising zinc ions. Combination compositions without Zinc ions (Composition B13) and with various concentrations of zinc ions (Compositions B14-B16) were inspected for appearance and turbidity after storage at 4° C./25° C./40° C. Compositions tested showed precipitation or turbidity over predetermined value (>3.0) after accelerated tests at 25° C./40° C. Compositions tested also showed turbidity over predetermined value (>1.5) after accelerated tests at 4° C.

### Example 4

The pharmacokinetics (PK) of an embodiment of the combination pharmaceutical composition of insulin aspart and GLP-1 (Beinaglutide) was investigated in beagles (male, 2-3 yr old, 9-11 kg).

After 11 beagles adapted to the laboratory environment, they were fasted for 20 hours without water, and then given food after 4 hours of drug administration. The beagles were randomly divided into three groups (n=3) according to their weight, and administered with GLP-1 only (20 μg/kg, 50 μL/kg), insulin aspart only (0.5 U/kg, 50 μL/kg), or a combination (50 μL/kg) of GLP-1 (10 μg/kg) and insulin aspart (0.5 U/kg).

Beagles were subjected to quick collection of 1.0 mL of whole blood using 1 mL syringe rinsed with 1 mL of DPPIV inhibitor and protease inhibitor before administration and after administration: for GLP-1 only group: 5, 10, 15, 20, 30, 40, 50, 60, 70, 80, 90 and 120 minutes post-administration, and for insulin aspart only group and the combination group, 5, 10, 15, 20, 30, 40, 50, 60, 70, 80, 90, 120, 180, 240, 300 and 480 minutes post-administration. The collected whole blood was added to an EDTA-K2 anticoagulant tube. The EDTA-K2 anticoagulant tube was pre-added with DPPIV inhibitor (10 uL) and protease inhibitor (10 uL). The obtained mixtures were placed in an ice bath within 30 min before centrifugation (4° C., 300 g, 5 min) to collect plasma. Samples of 0.3 mL plasma and 0.1 mL plasma were added respectively into two test tubes, store at −80° C., and were used for Active GLP-1 detection and Insulin Aspart detection respectively.

The concentration of insulin aspart and GLP-1 at each blood collection point were measured by Elisa kits. Parameters of PK were analyzed by PKSolver 2.0-compartmental analysis of the plasma data after extravascular input model (two-compartment).

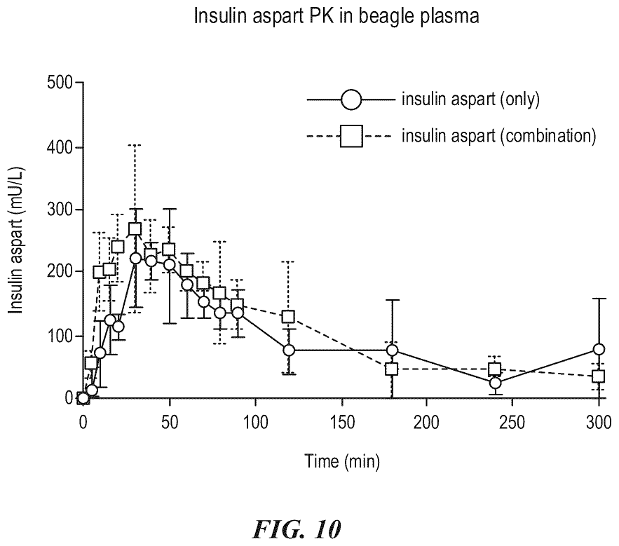

The insulin aspart concentrations changes over time are shown in FIG. 10. The parameters of PK are summarized in Table 4A.

TABLE 4A
Insulin aspart PK parameters

| | | Value | | |
|---|---|---|---|---|
| | | Insulin Aspart | Insulin Aspart | |
| Parameter | Unit | (Only) | (Combination) | Fold |
| Tₘₐₓ | min | 45.01 | 31.83 | 0.71 |
| Cₘₐₓ | pmol/L | 191.82 | 233.27 | 1.22 |
| AUC₀₋ₜ | pmol/L*min | 22837.96 | 29383.98 | 1.29 |

The Tₘₐₓ of the insulin spart only group was about 45 minutes, and the Tₘₐₓ of the combination group was about 32 minutes. The time to reach the peak concentration after administration of the combination was 13 minutes earlier than that of the insulin spart only administration, suggesting that insulin aspart had a faster onset of action in the combination administration than the insulin aspart only administration. The Cₘₐₓ and AUC₀₋ₜ of insulin aspart in the combination administration were 1.22 and 1.29 times of that of the insulin aspart only administration, respectively.

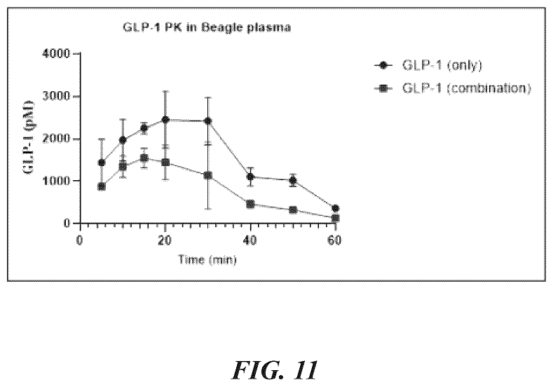

Similarly, the GLP-1 concentrations at each blood collection point in the GLP-1 only group and the combination group were measured and analyzed. The GLP-1 concentrations changes over time are shown in FIG. 11.

Considering that the dosage of GLP-1 in the GLP-1 only group was twice of the GLP-1 dosage in the combination group, the PK parameters were adjusted to the same dose of GLP-1 (10 ug/kg). The adjusted PK parameters are summarized in Table 4B:

TABLE 4B
GLP-1 PK parameters

| | | Value | | |
|---|---|---|---|---|
| | | GLP-1 | GLP-1 | |
| Parameter | Unit | (Only) | (Combination) | Fold |
| Tₘₐₓ | min | 16.67 | 13.80 | 0.83 |
| Cₘₐₓ | pmol/L | 1189.415 | 1471.89 | 1.24 |
| AUC₀₋ₜ | pmol/L*min | 47211.5 | 51425.77 | 1.09 |

The Tₘₐₓ of GLP-1 in the GLP-1 only group was about 17 minutes, and the Tₘₐₓ of GLP-1 in the combination group was about 14 minutes. The time to reach peak concentration after administration was slightly shorter in the combination group than that in the GLP-1 only group. The corrected Cₘₐₓ and AUC₀₋ₜ of the combination group were 1.24 and 1.09 times over that of the GLP-1 only group, respectively.

Beinaglutide (2 mg/ml, Acetate 10 mM, Mannitol 30 mg/ml, Phenol 2.2 mg/ml, Propylene Glycol 5 mg/ml, pH 4.0, Shanghai Benemae) was used for GLP-1.

Insulin aspart 100 U (3.5 mg)/ml, Glycerol 16 mg/ml, Phenol 1.5 mg/ml, M-Cresol 1.72 mg/ml, Disodium Phosphate Dibasic Dihydrate 1.25 mg/ml, Sodium Chloride 0.58 mg/ml, Zinc 19.6 g/ml, pH 7.4 Novo Nordisk) was used for insulin aspart.

Combination composition comprised insulin aspart (50 U/mL), GLP-1 (1.0 mg/mL), glycerol 20 mg/mL, phenol 3.2 mg/mL, zinc 20 μg/mL, pH 7.6, 0.2% DDM, 40 mM NaCl, and phosphate buffer (PB).

In the description of this specification, reference to the terms “one embodiment,” “some embodiments,” “an example,” “specific examples,” or “some examples” or the like means that specific features are described in connection with the embodiment or example, structures, materials or features are included in at least one embodiment or example of the invention. In this specification, schematic representations of the above terms do not necessarily refer to the same embodiment or example. Furthermore, the specific features, structures, materials or characteristics described may be combined in any suitable manner in any one or more embodiments or examples.

Although the embodiments of the present disclosure have been shown and described above, it can be understood that the above-mentioned embodiments are illustrative and should not be construed as limitations of the present disclosure. Those of ordinary skill in the art will not deviate from the principles and purposes of the present disclosure. Under the circumstances, the above-described embodiments can be changed, modified, replaced and modified within the scope of the present disclosure.

I/We claim:

1. A pharmaceutical composition comprising:
a first active ingredient comprising a GLP-1 receptor agonist;
one or more surfactants comprising one or more alkyl glycosides.

2. The pharmaceutical composition of claim 1, further comprising a second active ingredient comprising insulin, insulin analogues, and/or derivatives of the insulin analogues, optionally, the insulin analogue comprises at least one selected from insulin lispro, insulin aspart, insulin glulisine, insulin glargine, insulin degludec, or insulin detemir,
optionally, based on the total weight of the pharmaceutical composition, the pharmaceutical composition comprises 0.1˜1 wt % of the insulin or insulin analogues or derivatives of insulin analogues, and optionally, 0.1˜0.4 wt % of the insulin or insulin analogues or derivatives of insulin analogues.

3. The pharmaceutical composition of claim 1, wherein the alkyl glycoside comprises a sugar group and an alkyl group connected by a connecting group, wherein:
the sugar group comprises at least one sugar selected from glucose, maltose, sucrose, or trehalose;
the alkyl group comprises about 10 to about 16 carbon atoms;
and the connecting group comprises at least a bond selected from glycosidic bonds, thioglycosidic bonds, or amide bonds.

4. The pharmaceutical composition of claim 3, wherein the alkyl chain comprises 10˜14 carbon atoms.

5. The pharmaceutical composition of claim 3, wherein the sugar group comprises maltose or sucrose.

6. The pharmaceutical composition of claim 1, wherein the alkyl glycosides comprise at least a compound selected from n-dodecyl β-D-maltoside (DDM), sucrose monododecanoate, n-dodecyl β-D-maltoside, n-hexyl-β-D-glucoside, n-heptyl-β-D-glucoside, n-octyl-β-D-glucoside, n-nonyl-β-D-glucoside, n-decyl-β-D-glucoside, 3-cyclohexyl-1-propyl-β-D-glucoside, n-hexyl-β-D-glucopyranoside, n-octyl-β-D-maltoside, n-nonyl-β-D-maltoside, n-decyl-β-D-maltoside, cyclohexyl-methyl-β-D-maltoside, 2-cyclohexyl-ethyl-β-D-maltoside, 3-cyclohexyl-propyl-β-D-maltoside, 4-cyclohexyl-butyl-β-D-maltoside, or 5-cyclohexyl-amyl-β-D-maltoside,
optionally, based on the total weight of the pharmaceutical composition, the concentration of the alkyl glycosides is about 0.05% wt to about 0.50% wt or about 0.1% wt to about 0.20% wt.

7. The pharmaceutical composition of claim 1, wherein the GLP-1 receptor agonist is a peptide agonist.

8. The pharmaceutical composition of claim 7, wherein the GLP-1 receptor agonist is also a GIP agonist.

9. The pharmaceutical composition of claim 7, wherein the GLP-1 receptor agonist comprises at least one of the following:
GLP-1, GLP-1 analogues or derivatives of the GLP-1 analogues;
extended GLP-1 fragments or their analogues or derivatives; and
truncated GLP-1 fragments or their analogues or derivatives.

10. The pharmaceutical composition of claim 7, wherein the GLP-1 analogue comprises Exendin-4.

11. The pharmaceutical composition according to claim 1, wherein the GLP-1 receptor agonist includes at least one selected from the following: liraglutide, exenatide, lixisenatide, albiglutide, beinaglutide, dulaglutide, semaglutide, pepamotidide, and tilpotide.

12. The pharmaceutical composition according to claim 1, wherein the first active ingredient comprises or is beinaglutide and the second active ingredient comprises or is insulin glargine.

13. The pharmaceutical composition according to claim 1, wherein the first active ingredient comprises or is beinaglutide and the second active ingredient comprises or is insulin aspart.

14. The pharmaceutical composition according to any one of claim 1, wherein that the pharmaceutical composition is a liquid pharmaceutical composition, or a premixed aqueous injectable pharmaceutical composition.

15. The pharmaceutical composition according to claim 14, further comprising a bacteriostatic agent, optionally, the bacteriostatic agent comprises at least one compounds selected from phenolic compounds, aromatic alcohol compounds, phenoxyethanol, or chlorohydrin compounds.

16. The pharmaceutical composition according to claim 15, wherein the bacteriostatic agent comprises at least one of phenol, m-cresol, benzyl alcohol, phenoxyethanol or chlorobutanol.

17. The pharmaceutical composition according to claim 16, wherein the concentration of the GLP-1 receptor agonist is about 0.1 mg/mL to about 10 mg/mL.

18. The pharmaceutical composition of claim 16, wherein the pH of the pharmaceutical composition is about 3.0 to about 9.0,
optionally, the insulin analogue is insulin glargine, and the pH of the pharmaceutical composition is about 3.0 to about 5.0, and optionally, the pH of the is about 4.0 to about 4.5,
optionally, the insulin analogue is insulin aspart, and the pH of the pharmaceutical composition is about 7.0 to about 9.0.

19. The pharmaceutical composition of claim 16, wherein the pharmaceutical composition further comprises zinc ions, optionally, the concentration of zinc ions is about 1 μg/mL to about 100 μg/mL, about 10 μg/mL to about 50 μg/mL, about 20 μg/mL to about 40 μg/mL.

20. The pharmaceutical composition of claim 1, wherein the pharmaceutical composition further comprises a solubilizer comprising at least one of the following: glycerin, mannitol, propylene glycol, and combinations thereof, the concentration of the solubilizer is about 10 mg/mL to about 50 mg/mL based on the total volume of the pharmaceutical composition; preferably, the solubilizer is glycerin with a concentration of about 10 mg/mL to about 25 mg/mL based on the total volume of the pharmaceutical composition, or
the pharmaceutical composition does not comprise any of the following: polysorbate 20, polysorbate 80, poloxamer 188, hydroxypropyl betacyclodextrin and amino acids, optionally, the surfactant comprises only the alkyl glycosides.

21. The pharmaceutical composition of claim 1, wherein the pharmaceutical composition does not form a foreign object when it is in an accelerated stability test at 25° C. for 3 months, and the peptide % is not less than about 93%.

22. A method for improving the storage stability of a pharmaceutical composition comprising a GLP-1 receptor agonist, comprising mixing the pharmaceutical composition with one or more addition of alkyl glycosides.

23. The method of claim 22, wherein the pharmaceutical composition further comprises insulin or an insulin analogue or a derivative of the insulin analogue, and optionally, the insulin analogue comprises at least one of insulin lispro, insulin aspart, insulin glulisine, insulin glargine, insulin degludec, and insulin detemir.

24. The method of claim 22, wherein the alkyl glycoside comprises at least one of n-dodecyl β-D-maltoside (DDM), sucrose monododecanoate, n-dodecyl β-D-maltoside, n-hexyl-β-D-glucoside, n-heptyl-β-D-glucoside, n-octyl-β-D-glucoside, n-nonyl-β-D-glucoside, n-decyl-β-D-glucoside, 3-cyclohexyl-1-propyl-β-D-glucoside, n-hexyl-β-D-glucopyranoside, n-octyl-β-D-maltoside, n-nonyl-β-D-maltoside, n-decyl-β-D-maltoside, cyclohexyl-methyl-β-D-maltoside, 2-cyclohexyl-ethyl-β-D-maltoside, 3-cyclohexyl-propyl-β-D-maltoside, 4-cyclohexyl-butyl-β-D-maltoside, or 5-cyclohexyl-amyl-β-D-maltoside.

25. The use of the pharmaceutical composition according to any one of claim 1 for use in: (i) the prevention and/or treatment of diabetes, and/or for the reduction of HbA1C; (ii) the delay or prevention of the progression of diabetes, the delay of the progression of impaired glucose tolerance to insulin-requiring type 2 diabetes, the delay or prevention of insulin resistance, and/or the delay of the progression of type 2 diabetes that does not require insulin to insulin-requiring type 2 diabetes; (iii) the improvement of 3-cell function, and/or the restoration of 3-cell glucose sensitivity; (iv) the prevention and/or treatment of eating disorders; the reduction of gastric motility; the delay of gastric emptying; (v) the prevention and/or treatment of complications of obesity disease; (vi) the prevention and/or treatment of complications of diabetes; and/or (vii) the prevention and/or treatment of cardiovascular disease.


SEQUENCE LISTING (5 sequences)

SEQ ID NO: 1 - protein, 32 aa, synthetic construct
  REGION 1..32: note = Synthetic: GLP-1 receptor agonist
  VARIANT 1: note = X at position 1 is: R, or missing
  VARIANT 3: note = X at position 3 is: A, G, V, L, I, S, or T
  VARIANT 7: note = X at position 7 is: F, W, or Y
  VARIANT 11: note = X at position 11 is: V, S, W, I, L, K, F, or Y
  VARIANT 13: note = X at position 13 is: S, W, Y, F, K, I, L, or V
  VARIANT 14: note = X at position 14 is: Y, W, or F
  VARIANT 15: note = X at position 15 is: L, F, Y, or W
  VARIANT 17: note = X at position 17 is: G, E, D, Q, N, K, R, or C
  VARIANT 18: note = X at position 18 is: H, D, K, E, or Q
  VARIANT 20: note = X at position 20 is: A, V, I, or L
  VARIANT 21: note = X at position 21 is: K, R, Q, or N
  VARIANT 22: note = X at position 22 is: A, E, H, F, Y, W, R, I, or K
  VARIANT 25: note = X at position 25 is: A, E, D, S, or H
  VARIANT 28: note = X at position 28 is: V or I
  VARIANT 29: note = X at position 29 is: K, R, Q, or N
  VARIANT 31: note = X at position 31 is: R, R-NH2, K, or K-NH2
  VARIANT 32: note = X at position 32 is: G, K, R, T, S, E, D, W, Y, F, H, or missing
XHXEGTXTSDXSXXXEXXAXXXFIXWLXXGXX

SEQ ID NO: 2 - protein, 40 aa, synthetic construct
  REGION 1..40: note = Synthetic: GLP-1 receptor agonist
  VARIANT 1: note = X at position 1 is: H, R, Y, or EL
  VARIANT 2: note = X at position 2 is: S, G, A, or T
  VARIANT 3: note = X at position 3 is: D or E
  VARIANT 6: note = X at position 6 is: F or Y
  VARIANT 7: note = X at position 7 is: T, Y, or S
  VARIANT 8: note = X at position 8 is: S or Y
  VARIANT 9: note = X at position 9 is: D or E
  VARIANT 10: note = X at position 10 is: L or I
  VARIANT 14: note = X at position 14 is: L, I, V, or M
  VARIANT 20: note = X at position 20 is: R or K
  VARIANT 22: note = X at position 22 is: F or Y
  VARIANT 23: note = X at position 23 is: I, V, L, or M
  VARIANT 24: note = X at position 24 is: E or D
  VARIANT 25: note = X at position 25 is: W, F, or Y
  VARIANT 31: note = X at position 31 is: P or missing
  VARIANT 32: note = X at position 32 is: S or missing
  VARIANT 33: note = X at position 33 is: S or missing
  VARIANT 34: note = X at position 34 is: G or missing
  VARIANT 35: note = X at position 35 is: A or is missing
  VARIANT 36: note = X at position 36 is: P or missing
  VARIANT 37: note = X at position 37 is: P or missing
  VARIANT 38: note = X at position 38 is: P or missing
  VARIANT 39: note = X at position 39 is: S, R, or missing
  VARIANT 40: note = x at position 40 is any amino acid or missing
XXXGTXXXXXSKQXEEEAVXLXXXXLKNGGXXXXXXXXXX

SEQ ID NO: 3 - protein, 40 aa, synthetic construct
  REGION 1..40: note = Synthetic: P13 peptide sequence
ELAEGTFTSDLSKQMEEEAVRLFIEWLKNGGPSSGAPPPS

SEQ ID NO: 4 - protein, 31 aa, synthetic construct
  REGION 1..31: note = Synthetic: 988927 peptide sequence
HGEGTFTSDSSSYLEEQAAKEFIAWLVKGRG

SEQ ID NO: 5 - protein, 30 aa, synthetic construct
  REGION 1..30: note = Synthetic: GLP-1
HAEGTFTSDVSSYLEGQAAKEFIAWLVKGR